(19) United States
(12) Patent Application Publication
(10) Pub. No.: US 2024/0327494 A1
(43) Pub. Date: Oct. 3, 2024

(54) ACTRII PROTEINS AND USES THEREOF

(71) Applicant: Acceleron Pharma Inc., Cambridge, MA (US)

(72) Inventor: Janethe de Oliveira Pena, Cambridge, MA (US)

(21) Appl. No.: 18/580,425

(22) PCT Filed: Jul. 18, 2022
(86) PCT No.: PCT/US2022/037479

§ 371 Date: Jan. 18, 2024

Related U.S. Application Data
(60) Provisional application No. 63/223,265, filed on Jul. 19, 2021.

(51) Int. Cl. C07K 14/71; A61K 38/00

(52) U.S. Cl. CPC C07K 14/71; A61K 38/00; C07K 2319/30

(57) ABSTRACT

In some aspects, the disclosure relates to compositions and methods for decreasing a risk of telangiectasia in a patient receiving a therapeutically effective amount of an ActRII polypeptide, particularly a dosing regimen that decreases the risk of telangiectasia in a patient receiving a therapeutically effective amount of an ActRII polypeptide.

### CROSS-REFERENCE TO RELATED APPLICATIONS

This application claims the benefit of U.S. Provisional Application No. 63/223,265, filed Jul. 19, 2021, the content of which is hereby incorporated by reference in its entirety.

### FIELD

The present application describes a method of decreasing a risk of telangiectasia in a patient receiving a therapeutically effective amount of an ActRII polypeptide. The application also relates to a method of decreasing a risk of telangiectasia in a patient receiving a therapeutically effective amount of an ActRII polypeptide, wherein the polypeptide comprises an amino acid sequence that is at least 90% identical to an amino acid sequence corresponding to residues 30-110 of SEQ ID NO: 1.

### BACKGROUND

Telangiectasia refers to a condition in which widened venules (tiny blood vessels) cause threadlike red lines or patterns on the skin. These patterns, or telangiectases, are thought to be caused by the release or activation of vasoactive substances under a multitude of conditions. While telangiectases are often benign, they may be caused by a serious illness such as hereditary hemorrhagic telangiectasia (HHT). In patients with HHT, telangiectases may appear in vital organs, such as the liver. The rupture of these telangiectases may result in life-threatening hemorrhaging.

Telangiectasia may manifest as a side effect upon the administration of certain drugs, such as an ALK-1 receptor fusion protein (dalantercept), an endoglin-neutralizing antibody (TRC105), and certain ActRII polypeptides (Garber K. Nat Biotechnol. 2016; 34 (5): 458-461).

There is a high, unmet need for effective therapies for reducing the risk of telangiectasia in patients receiving one or more ActRII polypeptides for treatment of an underlying disease. Accordingly, it is an object of the present disclosure to provide methods for risk of telangiectasia in patients receiving an ActRII polypeptide.

### SUMMARY

In certain aspects, the disclosure provides a method of decreasing a risk of telangiectasia in a patient receiving a therapeutically effective amount of an ActRII polypeptide, wherein the method comprises administering the ActRII polypeptide on a dosing regimen comprising: (i) administering a first dose of ActRII polypeptide to a patient in an amount of 0.3 mg/kg; and (ii) administering a second dose of ActRII polypeptide to the patient in an amount of 0.7 mg/kg; wherein a third dose is initiated if the patient shows symptoms or comprises risk factors for developing telangiectasia, thereby decreasing the risk of telangiectasia. In certain aspects, the disclosure provides a method of decreasing a risk of telangiectasia in a patient receiving a therapeutically effective amount of an ActRII polypeptide, wherein the polypeptide comprises an amino acid sequence that is at least 90% identical to an amino acid sequence corresponding to residues 30-110 of SEQ ID NO: 1, wherein the method comprises administering the ActRII polypeptide on a dosing regimen comprising: (i) administering a first dose of ActRII polypeptide to a patient in an amount of 0.3 mg/kg; and (ii) administering a second dose of ActRII polypeptide to the patient in an amount of 0.7 mg/kg; wherein a third dose is initiated if the patient shows symptoms or comprises risk factors for developing telangiectasia, thereby decreasing the risk of telangiectasia. In some embodiments, the third dose comprises administering a dose of ActRII polypeptide to the patient in an amount of 0.3 mg/kg. In some embodiments, the third dose comprises withholding treatment of the ActRII polypeptide for a period of at least 2-6 weeks. In some embodiments, the third dose comprises withholding treatment of the ActRII polypeptide for a period of at least 3 weeks. In some embodiments, the first dose is administered to the patient for at least 3 weeks. In some embodiments, the second dose is administered for at least 21 weeks. In some embodiments, the second dose is administered for at least 45 weeks.

In certain aspects, the disclosure provides a method of reducing a risk of telangiesctasia when treating a patient with an ActRII polypeptide, wherein the method comprises administering an ActRII polypeptide on a dosing regimen comprising: (i) administering one or more first doses of the ActRII polypeptide to a patient in an amount of 0.3 mg/kg one every 3 weeks for a period of 24 weeks; wherein if the patient shows one or more symptoms or risk factors for developing telangiectasia administering one or more second doses of the ActRII polypeptide to the patient in an amount that is reduced by at least half of the amount of the first dose.

In certain aspects, the disclosure provides a method of decreasing a risk of telangiectasia in a patient receiving a therapeutically effective amount of an ActRII polypeptide wherein the method comprises administering the ActRII polypeptide on a dosing regimen comprising: (i) administering ActRII polypeptide to a patient in an amount of 0.3 mg/kg every 3 weeks; wherein if the patient shows symptoms or comprises risk factors for developing telangiectasia, administering ActRII polypeptide to a patient in an amount of 0.3 mg/kg every 6 weeks thereby decreasing the risk of telangiectasia. In certain aspects, the disclosure provides a method of decreasing a risk of telangiectasia in a patient receiving a therapeutically effective amount of an ActRII polypeptide, wherein the polypeptide comprises an amino acid sequence that is at least 90% identical to an amino acid sequence corresponding to residues 30-110 of SEQ ID NO: 1, wherein the method comprises administering the ActRII polypeptide on a dosing regimen comprising: (i) administering the ActRII polypeptide to a patient in an amount of 0.3 mg/kg every 3 weeks; wherein if the patient shows symptoms or comprises risk factors for developing telangiectasia, administering ActRII polypeptide to a patient in an amount of 0.3 mg/kg every 6 weeks thereby decreasing the risk of telangiectasia. In some embodiments, the therapeutically effective amount of the ActRII polypeptide is 0.7 mg/kg every 3 weeks.

In certain aspects, the disclosure provides a method of treating pulmonary arterial hypertension, wherein the method comprises administering to a patient in need thereof an ActRII polypeptide on a dosing regimen comprising: (i) administering a first dose of the ActRII polypeptide to the patient in an amount of 0.3 mg/kg; and (ii) administering a second dose of the ActRII polypeptide to the patient in an amount of 0.7 mg/kg once every 3 weeks for as long as the patient needs treatment; wherein the polypeptide comprises an amino acid sequence that is at least 90% identical to an amino acid sequence corresponding to residues 30-110 of SEQ ID NO: 1.

In some embodiments, the symptoms of telangiectasia are selected from the group consisting of pain, itching, threadlike red marks on the skin, mucocutaneous telangiectases, gastrointestinal bleeding, lesions on the skin, epistaxis, gingival bleeding, arteriovenous malformations, internal telangiectases, and red spots on the skin. In some embodiments, the symptoms of telangiectasia comprise lesions on the skin. In some embodiments, the symptoms of telangiectasia comprise gingival bleeding. In some embodiments, the symptoms of telangiectasia comprise epistaxis. In some embodiments, the symptoms of telangiectasia comprise arteriovenous malformations. In some embodiments, the symptoms of telangiectasia comprises internal telangiectases. In some embodiments, the arteriovenous malformations or internal telangiectases occur in internal organs (e.g., brain, liver, lungs, spleen, urinary tract, and spine). In some embodiments, the risk factors for developing telangiectasia are selected from the group consisting of: low BMP9 levels; low BMP10 levels; low VEGF levels; hereditary hemorrhagic telangiectasia (HHT); and connective tissue disease (CTD).

In some embodiments, the decreasing the risk comprises alleviating or improving the severity telangiectasia. In some embodiments, the decreasing the risk comprises preventing the progression of telangiectasia. In some embodiments, the third dose comprises withholding treatment of the ActRII polypeptide until the telangiectasia improves to ≤Grade 1. In some embodiments, the method improves the severity of telangiectasia from Grade 2 to Grade 1. In some embodiments, the method improves the severity of telangiectasia from Grade 3 to Grade 2. In some embodiments, the method improves the severity of telangiectasia from Grade 3 to Grade 1. In some embodiments, the method prevents progression of the telangiectasia from Grade 1 to Grade 2. In some embodiments, the method prevents progression of the telangiectasia from Grade 2 to Grade 3. In some embodiments, the patient is receiving the ActRII polypeptide for the treatment of PH. In some embodiments, the patient is receiving the ActRII polypeptide for the treatment of PAH.

In some embodiments, the polypeptide comprises an amino acid sequence that is at least 95% identical to an amino acid sequence corresponding to residues 30-110 of SEQ ID NO: 1. In some embodiments, the polypeptide comprises an amino acid sequence that is at least 90% identical to an amino acid sequence corresponding to residues 21-135 of SEQ ID NO: 1. In some embodiments, the polypeptide comprises an amino acid sequence that is at least 95% identical to an amino acid sequence corresponding to residues 21-135 of SEQ ID NO: 1. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 2. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3. In some embodiments, the ActRII polypeptide is a fusion protein further comprising an Fc domain of an immunoglobulin. In some embodiments, the Fc domain of the immunoglobulin is an Fc domain of an IgG1 immunoglobulin. In some embodiments, the Fc fusion protein further comprises a linker domain positioned between the ActRII polypeptide domain and the Fc domain of the immunoglobulin. In some embodiments, the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21). In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 40.

In some embodiments, the polypeptide is lyophilized. In some embodiments, the polypeptide is soluble. In some embodiments, the polypeptide is administered to the patient using subcutaneous injection. In some embodiments, the polypeptide is administered to the patient on a schedule selected from the group consisting of: every week, every 2 weeks, every 3 weeks, and every 4 weeks. In some embodiments, the polypeptide is administered to the patient every 3 weeks. In some embodiments, the polypeptide is administered to the patient every 4 weeks. In some embodiments, the polypeptide is part of a homodimer protein complex. In some embodiments, the polypeptide is glycosylated. In some embodiments, the polypeptide has a glycosylation pattern obtainable by expression in a Chinese hamster ovary cell. In some embodiments, the ActRII polypeptide binds to one or more ligands selected from the group consisting of: activin A, activin B, and GDF11. In some embodiments, the ActRII polypeptide binds to activin and/or GDF11. In some embodiments, the ActRII polypeptide further binds to one or more ligands selected from the group consisting of: BMP10, GDF8, and BMP6.

In some embodiments, the method further comprises administering to the patient an additional active agent and/or supportive therapy. In some embodiments, the additional active agent and/or supportive therapy is selected from the group consisting of: beta-blockers, angiotensin-converting enzyme inhibitors (ACE inhibitors), angiotensin receptor blockers (ARBs), diuretic agents, lipid-lowering medications, endothelin blockers, PDE5 inhibitors, prostacyclins, or a left ventricular assist device (LVAD). In some embodiments, the additional active agent and/or supportive therapy is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho [1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1→3)-beta-D-glucopyranosyl] oleanolic acid; 3-O-[beta-D-glucopyranosyl-(1→2)-beta-D-glucopyranosyl] oleanolic acid; 3-O-[beta-D-glucopyranosyl-(1→3)-beta-D-glucopyranosyl] oleanolic acid 28-O-beta-D-glucopyranosyl ester; 3-O-[beta-D-glucopyranosyl-(1→2)-beta-D-glucopyranosyl] oleanolic acid 28-O-beta-D-glucopyranosyl ester; 3-O-[a-L-rhamnopyranosyl-(1→3)-beta-D-glucuronopyranosyl] oleanolic acid; 3-O-[alpha-L-rhamnopyranosyl-(1→3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI4-2; Benzyl 3-dehydr-oxy-1,2,5-oxadiazolo [3′,4′: 2,3] oleanolate); a left ventricular assist device (LVAD), or lung and/or heart transplantation. In some embodiments, the patient has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the method further comprises administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil. In some embodiments, the patient has been treated with one or more vasodilators prior to administration of the polypeptide. In some embodiments, the method further comprises administration of one or more vasodilators. In some embodiments, the one or more vasodilators is selected from the group consisting of prostacyclin, epoprostenol, and sildenafil. In some embodiments, the vasodilator is prostacyclin. In some embodiments, the patient has been receiving one or more therapies for PAH. In some embodiments, the one or more therapies for PAH is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho [1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1→3)-beta-D-glucopyranosyl] oleanolic acid; 3-O-[beta-D-glucopyranosyl-(1→2)-beta-D-glucopyranosyl] oleanolic acid; 3-O-[beta-D-glucopyranosyl-(1→3)-beta-D-glucopyranosyl] oleanolic acid 28-O-beta-D-glucopyranosyl ester; 3-O-[beta-D-glucopyranosyl-(1→2)-beta-D-glucopyranosyl] oleanolic acid 28-O-beta-D-glucopyranosyl ester; 3-O-[a-L-rhamnopyranosyl-(1→3)-beta-D-glucuronopyranosyl] oleanolic acid; 3-O-[alpha-L-rhamnopyranosyl-(1→3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI4-2; Benzyl 3-dehydr-oxy-1,2,5-oxadiazolo [3′,4′: 2,3] oleanolate); a left ventricular assist device (LVAD), or lung and/or heart transplantation.

### BRIEF DESCRIPTION OF THE DRAWINGS

FIG. 1 shows an alignment of extracellular domains of human ActRIIB (SEQ ID NO: 31) and human ActRIIA (SEQ ID NO: 2) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIA proteins and human ActRIIA (SEQ ID NOs: 6-10 and 36-38).

FIG. 3 shows multiple sequence alignment of Fc domains from human IgG isotypes using Clustal 2.1. Hinge regions are indicated by dotted underline. Double underline indicates examples of positions engineered in IgG1 Fc (SEQ ID NO: 32) to promote asymmetric chain pairing and the corresponding positions with respect to other isotypes IgG2 (SEQ ID NO: 33), IgG3 (SEQ ID NO: 34) and IgG4 (SEQ ID NO: 35).

FIGS. 4A and 4B show the purification of ActRIIA-hFc expressed in CHO cells. The protein purifies as a single, well-defined peak as visualized by sizing column (FIG. 4A) and Coomassie stained SDS-PAGE (FIG. 4B) (left lane: molecular weight standards; right lane: ActRIIA-hFc).

FIGS. 5A and 5B show the binding of ActRIIA-hFc to activin (FIG. 5A) and GDF-11 (FIG. 5B), as measured by Biacore™ assay.

### DETAILED DESCRIPTION

### 1. Overview

The present disclosure relates to compositions and methods for decreasing a risk of telangiectasia in a patient receiving a therapeutically effective amount of an ActRII polypeptide as described herein, wherein the method comprises administering the ActRII polypeptide on a dosing regimen comprising: (i) administering a first dose of ActRII polypeptide to a patient in an amount of 0.3 mg/kg; and (ii) administering a second dose of ActRII polypeptide to the patient in an amount of 0.7 mg/kg; wherein a third dose is initiated if the patient shows symptoms or comprises risk factors for developing telangiectasia, thereby decreasing the risk of telangiectasia.

In certain embodiments, the present disclosure provides methods for decreasing a risk of telangiectasia in a patient receiving a therapeutically effective amount of an ActRII polypeptide for the treatment of pulmonary arterial hypertension (e.g., functional class II or functional class III), wherein the pulmonary arterial hypertension is treated by administering to a patient in need thereof an effective amount of an ActRII polypeptide as described herein.

Pulmonary arterial hypertension [World Health Organization (WHO) Group 1 PH] is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary artery pressures. These high pressures make it difficult for the heart to pump blood through the lungs to be oxygenated. Patients with PAH suffer from extreme shortness of breath as the heart struggles to pump against these high pressures. Patients with PAH typically develop significant increases in PVR and sustained elevations in mPAP, which ultimately lead to right ventricular failure and death. Patients diagnosed with PAH have a poor prognosis and equally compromised quality of life, with a mean life expectancy of 2 to 5 years from the time of diagnosis if untreated.

PAH can be diagnosed based on a mean pulmonary artery pressure of above 25 mmHg (or above 20 mmHg under updated guidelines) at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, ENG, SMAD9, CAV1, KCNK3, or EIF2AK4). In 70% of familial PAH cases, mutations are located in the BMPR2 gene. Risk factors for the development of PAH include family history of PAH, drug and toxin use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus). PAH may be associated with long term responders to calcium channel blockers, overt features of venous/capillaries (PVOD/PCH) involvement, and persistent PH of the newborn syndrome.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST (Basic Local Alignment Search Tool), BLAST-2, ALIGN, ALIGN-2, Clustal Omega, or Megalign (DNASTAR) software. Those skilled in the art can determine appropriate parameters for aligning sequences, including any algorithms needed to achieve maximal alignment over the full length of the sequences being compared. In some embodiments, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. Other algorithms for determining sequence identity or homology include: Clustal Omega, LALIGN, FASTA, SIM, and EMBOSS Needle. In a preferred embodiment, the algorithm used for determining sequence identity is Clustal Omega.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is +10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Polypeptides

In certain aspects, the disclosure relates to ActRII polypeptides and uses thereof (e.g., decreasing a risk of telangiectasia in a patient receiving a therapeutically effective amount of an ActRII polypeptide). As used herein, the term “ActRII” refers to the family of type II activin receptors. This family includes activin receptor type IIA (ActRIIA) and activin receptor type IIB (ActRIIB).

In certain embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in anyone of SEQ ID NOs: 1, 2, 3, 23, 27, 30, and 40. In other embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in SEQ ID NO: 31. As used herein, the term “ActRII” refers to a family of activin receptor type IIA (ActRIIA) proteins, a family of activin receptor type IIB (ActRIIB) proteins, or combinations and/or variants thereof. The ActRII polypeptides can be derived from any species and include variants derived from such ActRII proteins by mutagenesis or other modification. Reference to ActRII herein is understood to be a reference to any one of the currently identified forms. Members of the ActRII family are generally transmembrane proteins, composed of a ligand-binding extracellular domain comprising a cysteine-rich region, a transmembrane domain, and a cytoplasmic domain with predicted serine/threonine kinase activity.

The term ActRII polypeptide includes polypeptides comprising any naturally occurring polypeptide of an ActRII family member as well as any variants thereof (including mutants, fragments, fusions, and peptidomimetic forms) that retain a useful activity. Examples of such variant ActRII polypeptides are provided throughout the present disclosure as well as in International Patent Application Publication Nos. WO 2006/012627, WO 2007/062188, WO 2008/097541, WO 2010/151426, and WO 2011/020045, which are incorporated herein by reference in their entirety. Numbering of amino acids for all ActRII-related polypeptides described herein is based on the numbering of the human ActRII precursor protein sequence provided below (SEQ ID NO: 1), unless specifically designated otherwise.

The canonical human ActRII precursor protein sequence is as follows:

| (SEQ ID NO: 1) | |
|---|---|
| 1 | MGAAAKLAFA VFLISCSSGA ILGRSETQEC LFFNANWEKD RT [symbol] NQTGVEPC |
| 51 | YGDKDKRRHC FATWK [symbol] ISGS IEIVKQGCWL DDINCYDRTD CVEKKDSPEV |
| 101 | YFCCCEGNMC NEKFSYFPEM EVTQPTSNPV TPKPPYYNIL LYSLVPLMLI |
| 151 | AGIVICAFWV YRHHKMAYPP VLVPTQDPGP PPPSPLLGLK PLQLLEVKAR |
| 201 | GRFGCVWKAQ LLNEYVAVKI FPIQDKQSWQ NEYEVYSLPG MKHENILQFI |
| 251 | GAEKRGTSVD VDLWLITAFH EKGSLSDFLK ANVVSWNELC HIAETMARGL |
| 301 | AYLHEDIPGL KDGHKPAISH RDIKSKNVLL KNNLTACIAD FGLALKFEAG |
| 351 | KSAGDTHGQV GTRRYMAPEV LEGAINFQRD AFLRIDMYAM GLVLWELASR |
| 401 | CTAADGPVDE YMLPFEEEIG QHPSLEDMQE VVVHKKKRPV LRDYWQKHAG |
| 451 | MAMLCETIEE CWDHDAEARL SAGCVGERIT QMQRLINIIT TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed (mature) extracellular human ActRII polypeptide sequence is as follows:

(SEQ ID NO: 2)
ILGRSETQECLFFNANWEKDRINQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPP

The C-terminal “tail” of the extracellular domain is indicated by single underline. The sequence with the “tail” deleted (a 415 sequence) is as follows:
ILGRSETQECLFFNANWEKDRTNQTGVEPCYGDKDKRRHCFATWKNISGSIEIVKQGCWLDD INCYDRTDCVEKKDSPEVYFCCCEGNMCNEKESYFPEM (SEQ ID NO: 3)

The nucleic acid sequence encoding human ActRII precursor protein is shown below (SEQ ID NO: 4), as follows nucleotides 159-1700 of Genbank Reference Sequence NM_001616.4. The signal sequence is underlined.

| (SEQ ID NO: 4) | |
|---|---|
| 1 | ATGGGAGCTG CTGCAAAGTT GGCGTTTGCC GTCTTTCTTA TCTCCTGTTC |
| 51 | TTCAGGTGCT ATACTTGGTA GATCAGAAAC TCAGGAGTGT CTTTTCTTTA |
| 101 | ATGCTAATTG GGAAAAAGAC AGAACCAATC AAACTGGTGT TGAACCGTGT |
| 151 | TATGGTGACA AAGATAAACG GCGGCATTGT TTTGCTACCT GGAAGAATAT |
| 201 | TTCTGGTTCC ATTGAAATAG TGAAACAAGG TTGTTGGCTG GATGATATCA |
| 251 | ACTGCTATGA CAGGACTGAT TGTGTAGAAA AAAAAGACAG CCCTGAAGTA |
| 301 | TATTTTTGTT GCTGTGAGGG CAATATGTGT AATGAAAAGT TTTCTTATTT |
| 351 | TCCGGAGATG GAAGTCACAC AGCCCACTTC AAATCCAGTT ACACCTAAGC |
| 401 | CACCCTATTA CAACATCCTG CTCTATTCCT TGGTGCCACT TATGTTAATT |
| 451 | GCGGGGATTG TCATTTGTGC ATTTTGGGTG TACAGGCATC ACAAGATGGC |
| 501 | CTACCCTCCT GTACTTGTTC CAACTCAAGA CCCAGGACCA CCCCCACCTT |
| 551 | CTCCATTACT AGGTTTGAAA CCACTGCAGT TATTAGAAGT GAAAGCAAGG |
| 601 | GGAAGATTTG GTTGTGTCTG GAAAGCCCAG TTGCTTAACG AATATGTGGC |
| 651 | TGTCAAAATA TTTCCAATAC AGGACAAACA GTCATGGCAA AATGAATACG |
| 701 | AAGTCTACAG TTTGCCTGGA ATGAAGCATG AGAACATATT ACAGTTCATT |
| 751 | GGTGCAGAAA AACGAGGCAC CAGTGTTGAT GTGGATCTTT GGCTGATCAC |
| 801 | AGCATTTCAT GAAAAGGGTT CACTATCAGA CTTTCTTAAG GCTAATGTGG |
| 851 | TCTCTTGGAA TGAACTGTGT CATATTGCAG AAACCATGGC TAGAGGATTG |
| 901 | GCATATTTAC ATGAGGATAT ACCTGGCCTA AAAGATGGCC ACAAACCTGC |
| 951 | CATATCTCAC AGGGACATCA AAAGTAAAAA TGTGCTGTTG AAAAACAACC |
| 1001 | TGACAGCTTG CATTGCTGAC TTTGGGTTGG CCTTAAAATT TGAGGCTGGC |
| 1051 | AAGTCTGCAG GCGATACCCA TGGACAGGTT GGTACCCGGA GGTACATGGC |
| 1101 | TCCAGAGGTA TTAGAGGGTG CTATAAACTT CCAAAGGGAT GCATTTTTGA |
| 1151 | GGATAGATAT GTATGCCATG GGATTAGTCC TATGGGAACT GGCTTCTCGC |
| 1201 | TGTACTGCTG CAGATGGACC TGTAGATGAA TACATGTTGC CATTTGAGGA |
| 1251 | GGAAATTGGC CAGCATCCAT CTCTTGAAGA CATGCAGGAA GTTGTTGTGC |
| 1301 | ATAAAAAAAA GAGGCCTGTT TTAAGAGATT ATTGGCAGAA ACATGCTGGA |
| 1351 | ATGGCAATGC TCTGTGAAAC CATTGAAGAA TGTTGGGATC ACGACGCAGA |
| 1401 | AGCCAGGTTA TCAGCTGGAT GTGTAGGTGA AAGAATTACC CAGATGCAGA |
| 1451 | GACTAACAAA TATTATTACC ACAGAGGACA TTGTAACAGT GGTCACAATG |
| 1501 | GTGACAAATG TTGACTTTCC TCCCAAAGAA TCTAGTCTA |

The nucleic acid sequence encoding processed soluble (extracellular) human ActRII polypeptide is as follows:

| (SEQ ID NO: 5) | |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

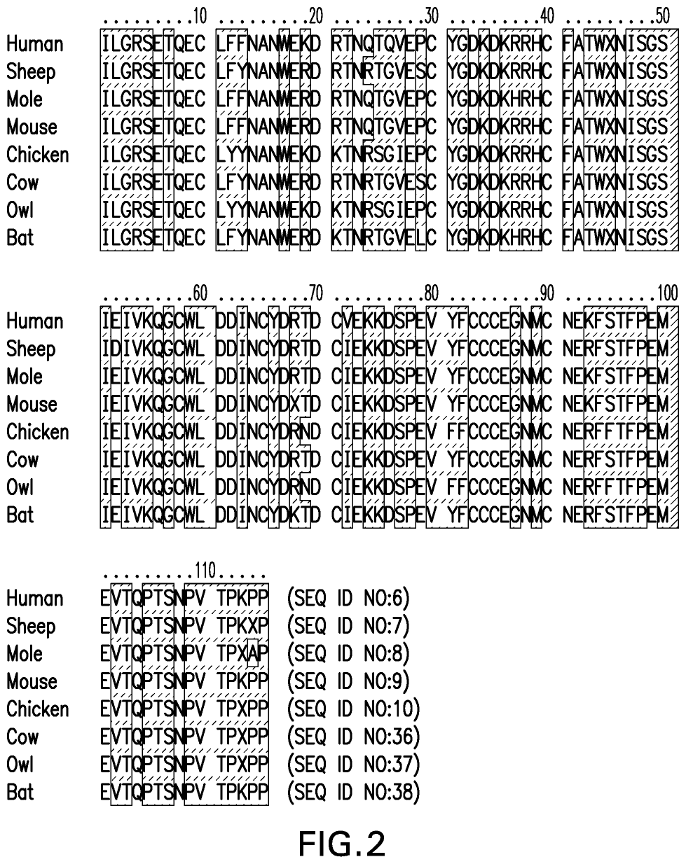

ActRII is well-conserved among vertebrates, with large stretches of the extracellular domain completely conserved. For example, FIG. 2 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRII-ligand binding activities as well as to predict amino acid positions that are likely to be tolerant to substitution without significantly altering normal ActRII-ligand binding activities. Therefore, an active, human ActRII variant polypeptide useful in accordance with the presently disclosed methods may include one or more amino acids at corresponding positions from the sequence of another vertebrate ActRII, or may include a residue that is similar to that in the human or other vertebrate sequences.

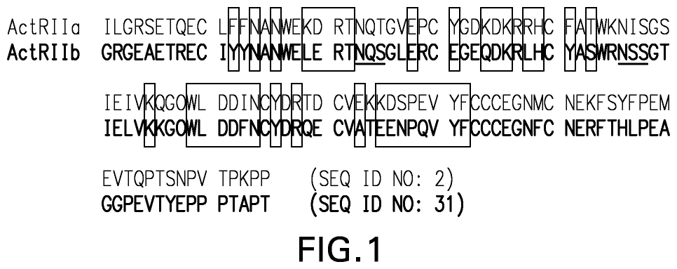

An alignment of the amino acid sequences of human ActRIIA extracellular domain and human ActRIIB extracellular domain are illustrated in FIG. 1. This alignment indicates amino acid residues within both receptors that are believed to directly contact ActRII ligands. For example, the composite ActRII structures indicated that the ActRIIA-ligand binding pocket is defined, in part, by residues F31, N33, N35, K38 through T41, E47, Y50, K53 through K55, R57, H58, F60, T62, K74, W78 through N83, Y85, R87, E92, and K94 through F101. At these positions, it is expected that conservative mutations will be tolerated.

Without meaning to be limiting, the following examples illustrate this approach to defining an active ActRII variant. As illustrated in FIG. 2, F13 in the human extracellular domain is Y in Ovis aries (SEQ ID NO: 7), Gallus gallus (SEQ ID NO: 10), Bos Taurus (SEQ ID NO: 36), Tyto alba (SEQ ID NO: 37), and Myotis davidii (SEQ ID NO: 38) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68 (1): 97-108; Greenwald et al. (1999) Nature Structural Biology 6 (1): 18-22; Allendorph et al. (2006) PNAS 103 (20:7643-7648; Thompson et al. (2003) The EMBO Journal 22 (7): 1555-1566; as well as U.S. Pat. Nos. 7,709,605, 7,612,041, and 7,842,663]. For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. In addition to the teachings herein, these references provide amply guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRII, as demarcated by the outermost of these conserved cysteines, corresponds to positions 30-110 of SEQ ID NO: 1 (ActRII precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRII extracellular domains truncations include SEQ ID NOs: 2 and 3.

Accordingly, a general formula for an active portion (e.g., ligand binding) of ActRII is a polypeptide that comprises, consists essentially of, or consists of amino acids 30-110 of SEQ ID NO: 1. Therefore ActRII polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 1. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 1, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1. Thus, in some embodiments, an ActRII polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1. Optionally, ActRII polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1, and comprising no more than 1, 2, 5, 10 or 15 conservative amino acid changes in the ligand-binding pocket. In some embodiments, the ActRII polypeptide is part of a homodimer protein complex.

In certain embodiments, the disclosure relates to an ActRII polypeptide (e.g., ActRIIA polypeptides, ActRIIB polypeptides, or combinations thereof), which includes fragments, functional variants, and modified forms thereof as well as uses thereof (e.g., decreasing a risk of telangiectasia in a patient receiving a therapeutically effective amount of an ActRII polypeptide). Preferably, ActRII polypeptides are soluble (e.g., an extracellular domain of ActRII). In some embodiments, ActRII polypeptides inhibit (e.g., Smad signaling) of one or more GDF/BMP ligands [e.g., GDF11, GDF8, activin A, activin B, GDF3, BMP4, BMP6, BMP10, and/or BMP15]. In some embodiments, ActRII polypeptides bind to one or more GDF/BMP ligands [e.g., GDF11, GDF8, activin A, activin B, GDF3, BMP4, BMP6, BMP10, and/or BMP15]. In some embodiments, ActRII polypeptide of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 1. In certain embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 23, 27, 30, and 40.

In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some alternative embodiments, the ActRII polypeptide (e.g., SEQ ID NO: 23) may lack the C-terminal lysine. In some embodiments, the ActRII polypeptide lacking the C-terminal lysine is SEQ ID NO: 40. In some embodiments, the ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 40. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 40. In some embodiments, a patient is administered a combination of SEQ ID NO: 23 and SEQ ID NO: 40.

In certain aspects, the present disclosure relates to ActRII polypeptides (e.g., ActRIIA polypeptides, ActRIIB polypeptides, or combinations thereof). In some embodiments, ActRII traps of the present disclosure are variant ActRII polypeptides (e.g., ActRIIA polypeptides, ActRIIB polypeptides, or combinations thereof) that comprise one or more mutations (e.g., amino acid additions, deletions, substitutions, and combinations thereof) in the extracellular domain (also referred to as the ligand-binding domain) of an ActRII polypeptide (e.g., a “wild-type” or unmodified ActRII polypeptide) such that the variant ActRII polypeptide has one or more altered ligand-binding activities than the corresponding wild-type ActRII polypeptide. In preferred embodiments, variant ActRII polypeptides of the present disclosure retain at least one similar activity as a corresponding wild-type ActRII polypeptide. For example, preferable ActRII polypeptides bind to and inhibit (e.g. antagonize) the function of GDF11 and/or GDF8. In some embodiments, ActRII polypeptides of the present disclosure further bind to and inhibit one or more of ligand of the GDF/BMP [e.g., GDF11, GDF8, activin A, activin B, GDF3, BMP4, BMP6, BMP10, and/or BMP15]. Accordingly, the present disclosure provides ActRII polypeptides that have an altered binding specificity for one or more ActRII ligands.

To illustrate, one or more mutations may be selected that increase the selectivity of the altered ligand-binding domain for GDF11 and/or GDF8 over one or more ActRII-binding ligands such as activins (activin A or activin B), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

In certain embodiments, the present disclosure contemplates specific mutations of an ActRII polypeptide (e.g., ActRIIA polypeptides, ActRIIB polypeptides, or combinations thereof) so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ActRII polypeptide (e.g., ActRIIA polypeptides, ActRIIB polypeptides, or combinations thereof) as well as truncation mutants. Pools of combinatorial mutants are especially useful for identifying functionally active (e.g., GDF/BMP ligand binding) ActRII sequences. The purpose of screening such combinatorial libraries may be to generate, for example, polypeptides variants, which have altered properties, such as altered pharmacokinetic or altered ligand binding. A variety of screening assays are provided below, and such assays may be used to evaluate variants. For example, ActRII variants may be screened for ability to bind to one or more GDF/BMP ligands [e.g., GDF11, GDF8, activin A, activin B, GDF3, BMP4, BMP6, BMP10, and/or BMP15], to prevent binding of a GDF/BMP ligand to an ActRII polypeptide, as well as heteromultimers thereof, and/or to interfere with signaling caused by an GDF/BMP ligand.

The activity of ActRII polypeptides (e.g., ActRIIA polypeptides, ActRIIB polypeptides, or combinations thereof) or variants thereof may also be tested in a cell-based or in vivo assay. For example, the effect of an ActRII polypeptide on the expression of genes involved in pulmonary arterial hypertension pathogenesis may be assessed. This may, as needed, be performed in the presence of one or more recombinant ligand proteins [e.g., GDF11, GDF8, activin A, activin B, GDF3, BMP4, BMP6, BMP10, and/or BMP15], and cells may be transfected so as to produce an ActRII polypeptide, and optionally, an GDF/BMP ligand. Likewise, an ActRII polypeptide may be administered to a mouse or other animal and effects on pulmonary arterial hypertension pathogenesis may be assessed using art-recognized methods. Similarly, the activity of an ActRII polypeptide or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference ActRII polypeptide (e.g., ActRIIA polypeptides, ActRIIB polypeptides, or combinations thereof). Such variants, when expressed from recombinant DNA constructs, can be used in gene therapy protocols. Likewise, mutagenesis can give rise to variants which have intracellular half-lives dramatically different than the corresponding unmodified ActRII polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the ActRII polypeptide.

A combinatorial library may be produced by way of a degenerate library of genes encoding a library of polypeptides which each include at least a portion of potential ActRII polypeptide sequences. For instance, a mixture of synthetic oligonucleotides can be enzymatically ligated into gene sequences such that the degenerate set of potential ActRII encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249:404-406; Cwirla et al., (1990) PNAS USA 87:6378-6382; as well as U.S. Pat. Nos. 5,223,409, 5,198,346, and 5,096,815].

Alternatively, other forms of mutagenesis can be utilized to generate a combinatorial library. For example, ActRII polypeptides of the disclosure (e.g., ActRIIA polypeptides, ActRIIB polypeptides, or combinations thereof) can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; Mcknight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of ActRII polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRII polypeptides (e.g., ActRIIA polypeptides, ActRIIB polypeptides, or combinations thereof). The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include ligand [e.g., GDF11, GDF8, activin A, activin B, GDF3, BMP4, BMP6, BMP10, and/or BMP15] binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of ActRII polypeptides (e.g., ActRIIA polypeptides, ActRIIB polypeptides, or combinations thereof) that can be used as guidance for generating and using other variant ActRII polypeptides within the scope of the disclosure provided herein.

In certain embodiments, functionally active fragments of ActRII polypeptides of the present disclosure can be obtained by screening polypeptides recombinantly produced from the corresponding fragment of the nucleic acid encoding an ActRII polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII receptors and/or one or more ligands [e.g., GDF11, GDF8, activin A, activin B, GDF3, BMP4, BMP6, BMP10, and/or BMP15].

In certain embodiments, ActRII polypeptides of the present disclosure (e.g., ActRIIA polypeptides, ActRIIB polypeptides, or combinations thereof) may further comprise post-translational modifications in addition to any that are naturally present in the ActRII polypeptide. Such modifications include, but are not limited to, acetylation, carboxylation, glycosylation, phosphorylation, lipidation, and acylation. As a result, the ActRII polypeptide may contain non-amino acid elements, such as polyethylene glycols, lipids, polysaccharide or monosaccharide, and phosphates. Effects of such non-amino acid elements on the functionality of a ligand trap polypeptide may be tested as described herein for other ActRII variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the ActRII polypeptides.

In certain aspects, ActRII polypeptides of the present disclosure (e.g., ActRIIA polypeptides, ActRIIB polypeptides, or combinations thereof) include fusion proteins having at least a portion (domain) of an ActRII polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) (SEQ ID NO: 39) fusion partners. As another example, a fusion domain may be selected so as to facilitate detection of the ActRII polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, ActRII polypeptides of the present disclosure (e.g., ActRIIA polypeptides, ActRIIB polypeptides, or combinations thereof) contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising an ActRII polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol. In certain preferred embodiments, an ActRII polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., a Fc domain) are known to confer desirable pharmacokinetic properties on a wide range of proteins. Likewise, fusions to human serum albumin can confer desirable properties.

An example of a native amino acid sequence that may be used for the Fc portion of human IgG1 (G1Fc) is shown below (SEQ ID NO: 11). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 11. Naturally occurring variants in G1Fc would include E134D and M136L according to the numbering system used in SEQ ID NO: 11 (see Uniprot P01857).

(SEQ ID NO: 11)

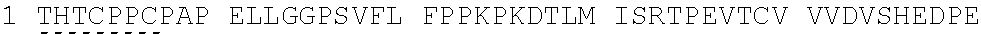

51 VKFNWYVDGV EVHNAKTKPR EEQYNSTYRV VSVLTVLHQD WLNGKEYKCK
101 VSNKALPAPI EKTISKAKGQ PREPQVYTLP PSREEMTKNQ VSLTCLVKGF
151 YPSDIAVEWE SNGQPENNYK TTPPVLDSDG SFFLYSKLTV DKSRWQQGNV
201 FSCSVMHEAL HNHYTOKSLS LSPGK

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 12). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 12.

(SEQ ID NO: 12)

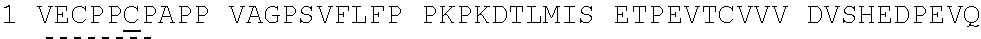

51 FNWYVDGVEV HNAKTKPREE QFNSTERVVS VLTVVHODWL NGKEYKCKVS
101 NKGLPAPIEK TISKTKGQPR EPQVYTLPPS REEMTKNQVS LTCLVKGFYP
151 SDIAVEWESN GQPENNYKTT PPMLDSDGSF FLYSKLTVDK SRWQQGNVES
201 CSVMHEALHN HYTQKSLSLS PGK

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 13) contains a short hinge region consisting of a single 15-residue segment, whereas the second G3Fc sequence (SEQ ID NO: 14) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 13 and 14.

(SEQ ID NO: 13)

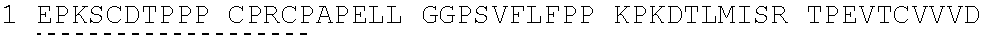

51 VSHEDPEVQF KWYVDGVEVH NAKTKPREEQ YNSTERVVSV LTVLHQDWLN
101 GKEYKCKVSN KALPAPIEKT ISKTKGQPRE PQVYTLPPSR EEMTKNQVSL
151 TCLVKGFYPS DIAVEWESSG QPENNYNTTP PMLDSDGSFF LYSKLTVDKS
201 RWQQGNIFSC SVMHEALHNR FTQKSLSLSP GK
(SEQ ID NO: 14)

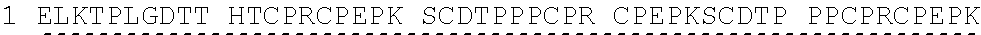

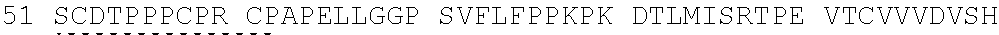

101 EDPEVOFKWY VDGVEVHNAK TKPREEQYNS TFRVVSVLTV LHQDWLNGKE
151 YKCKVSNKAL PAPIEKTISK TKGQPREPQV YTLPPSREEM TKNQVSLTCL
201 VKGFYPSDIA VEWESSGQPE NNYNTIPPML DSDGSFFLYS KLTVDKSRWQ
251 QGNIFSCSVM HEALHNRFTQ KSLSLSPGK

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 13, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CHI region. It has an extra interchain disulfide bond at position 7 in addition to the 11 normally present in the hinge region. Variant ZUC lacks most of the V region, all of the CHI region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 15). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15.

(SEQ ID NO: 15)

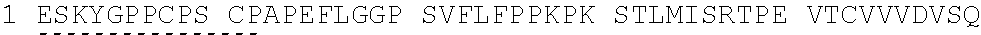

51 EDPEVQFNWY VDGVEVHNAK TKPREEQENS TYRVVSVLIV LHQDWLNGKE
101 YKCKVSNKGL PSSIEKTISK AKGQPREPQV YTLPPSQEEM TKNQVSLTCL
151 VKGFYPSDIA VEWESNGQPE NNYKTTPPVL DSDGSFFLYS RLTVDKSRWQ
201 EGNVFSCSVM HEALHNHYTQ KSLSLSLGK

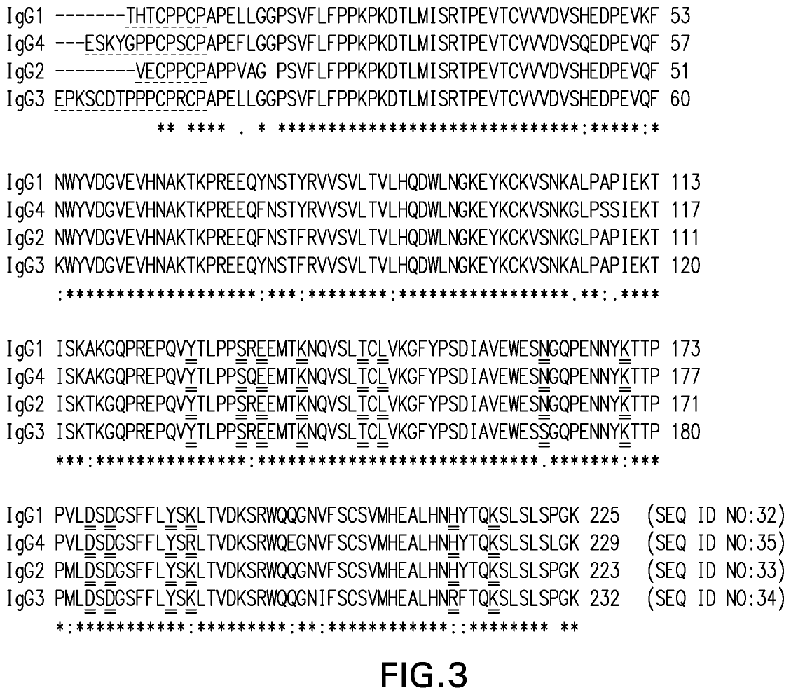

A variety of engineered mutations in the Fc domain are presented herein with respect to the G1Fc sequence (SEQ ID NO: 11), and analogous mutations in G2Fc, G3Fc, and G4Fc can be derived from their alignment with G1Fc in FIG. 3. Due to unequal hinge lengths, analogous Fc positions based on isotype alignment (FIG. 3) possess different amino acid numbers in SEQ ID NOs: 11, 12, 13, 14, and 15. It can also be appreciated that a given amino acid position in an immunoglobulin sequence consisting of hinge, C_{H}2, and C_{H}3 regions (e.g., SEQ ID NOs: 11, 12, 13, 14, and 15) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 11), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | |
|---|---|---|
| (Numbering begins | constant domain | IgG1 heavy chain |
| at first threonine | (Numbering begins at | (EU numbering scheme |
| in hinge region) | C_{H}1) | of Kabat et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

Various methods are known in the art that increase desired pairing of Fc-containing fusion polypeptide chains in a single cell line to produce a preferred asymmetric fusion protein at acceptable yields [Klein et al (2012) mAbs 4:653-663; and Spiess et al (2015) Molecular Immunology 67 (2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing [Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605].

It is understood that different elements of the fusion proteins (e.g., immunoglobulin Fc fusion proteins) may be arranged in any manner that is consistent with desired functionality. For example, an ActRII polypeptide domain may be placed C-terminal to a heterologous domain, or alternatively, a heterologous domain may be placed C-terminal to an ActRII polypeptide domain. The ActRII polypeptide domain and the heterologous domain need not be adjacent in a fusion protein, and additional domains or amino acid sequences may be included C- or N-terminal to either domain or between the domains.

For example, an ActRII receptor fusion protein may comprise an amino acid sequence as set forth in the formula A-B-C. The B portion corresponds to an ActRII polypeptide domain (e.g., ActRIIA polypeptides, ActRIIB polypeptides, or combinations thereof). The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 16), GGGG (SEQ ID NO: 17), TGGGG (SEQ ID NO: 18), SGGGG (SEQ ID NO: 19), TGGG (SEQ ID NO: 20), SGGG (SEQ ID NO: 21), or GGGGS (SEQ ID NO: 22) singlets, or repeats. In certain embodiments, an ActRII fusion protein comprises an amino acid sequence as set forth in the formula A-B-C, wherein A is a leader (signal) sequence, B consists of an ActRII polypeptide domain, and C is a polypeptide portion that enhances one or more of in vivo stability, in vivo half-life, uptake/administration, tissue localization or distribution, formation of protein complexes, and/or purification. In certain embodiments, an ActRII fusion protein comprises an amino acid sequence as set forth in the formula A-B-C, wherein A is a TPA leader sequence, B consists of an ActRII receptor polypeptide domain, and C is an immunoglobulin Fc domain. Preferred fusion proteins comprise the amino acid sequence set forth in any one of SEQ ID NOs: 23, 27, 30, and 40.

In preferred embodiments, ActRII polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides to be used in accordance with the methods described herein are recombinant polypeptides.

ActRII polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length ActRII polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ActRII polypeptides using chemical cleavage (e.g., cyanogen bromide, hydroxylamine, etc.).

### 3. Nucleic Acids Encoding ActRII Polypeptides

In certain embodiments, the present disclosure provides isolated and/or recombinant nucleic acids encoding ActRII polypeptides (e.g., ActRIIA polypeptides, ActRIIB polypeptides, or combinations thereof) including fragments, functional variants, and fusion proteins thereof.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII polypeptides of the disclosure are understood to include nucleic acids that are variants of any one of SEQ ID NOs: 4, 5, or 28. Variant nucleotide sequences include sequences that differ by one or more nucleotide substitutions, additions, or deletions including allelic variants, and therefore, will include coding sequence that differ from the nucleotide sequence designated in any one of SEQ ID NOs: 4, 5, or 28.

In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to any one of SEQ ID NOs: 4, 5, or 28. One of ordinary skill in the art will appreciate that nucleic acid sequences that are at least 70%, 75%, 80%, 85%, 90%, 91%, 92%, 93%, 94% 95%, 96%, 97%, 98%, 99%, or 100% identical to the sequences complementary to SEQ ID NOs: 4, 5, or 28, and variants thereof, are also within the scope of the present disclosure. In further embodiments, the nucleic acid sequences of the disclosure can be isolated, recombinant, and/or fused with a heterologous nucleotide sequence, or in a DNA library.

In other embodiments, nucleic acids of the present disclosure also include nucleotide sequences that hybridize under highly stringent conditions to the nucleotide sequence designated in SEQ ID NOs: 4, 5, or 28, complement sequences of SEQ ID NOs: 4, 5, or 28, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0 × sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOS: 4, 5, or 28 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art and can be used in a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and can vary with the host cell used.

In certain aspects, the subject nucleic acid disclosed herein is provided in an expression vector comprising a nucleotide sequence encoding an ActRII polypeptide (e.g., ActRIIA polypeptides, ActRIIB polypeptides, or combinations thereof) operably linked to at least one regulatory sequence. Regulatory sequences are art-recognized and are selected to direct expression of the ActRII polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding an ActRII polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In a preferred embodiment, a vector will be designed for production of the subject ActRII polypeptides in CHO cells, such as a Pemv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ActRII polypeptides in cells propagated in culture, e.g., to produce proteins, including fusion proteins or variant proteins, for purification.

This disclosure also pertains to a host cell transfected with a recombinant gene including a coding sequence for one or more of the subject ActRII polypeptides. The host cell may be any prokaryotic or eukaryotic cell. For example, an ActRII polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject ActRII polypeptides. For example, a host cell transfected with an expression vector encoding an ActRII polypeptide can be cultured under appropriate conditions to allow expression of the ActRII polypeptide to occur. The polypeptide may be secreted and isolated from a mixture of cells and medium containing the polypeptide. Alternatively, the ActRII polypeptide may be retained cytoplasmically or in a membrane fraction and the cells harvested, lysed and the protein isolated. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRII polypeptides, and affinity purification with an agent that binds to a domain fused to the ActRII polypeptide (e.g., a protein A column may be used to purify an ActRII-Fc fusion proteins). In some embodiments, the ActRII polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII polypeptide, can allow purification of the expressed fusion protein by affinity chromatography using a Ni²⁺ metal resin. The purification leader sequence can then be subsequently removed by treatment with enterokinase to provide the purified ActRII polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 4. Methods of Use

In part, the present disclosure relates to methods of decreasing a risk of telangiectasia in a patient receiving a therapeutically effective amount of an ActRII polypeptide described herein, wherein the method comprises administering the ActRII polypeptide on a dosing regimen comprising: (i) administering a first dose of ActRII polypeptide to a patient in an amount of 0.3 mg/kg; and (ii) administering a second dose of ActRII polypeptide to the patient in an amount of 0.7 mg/kg; wherein a third dose is initiated if the patient shows symptoms or comprises risk factors for developing telangiectasia, thereby decreasing the risk of telangiectasia. In some embodiments, the patient is receiving a therapeutically effective amount of an ActRII polypeptide described herein for the treatment of pulmonary arterial hypertension (PAH).

These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating” and the like are used herein to generally mean obtaining a desired pharmacologic and/or physiologic effect, and may also be used to refer to improving, alleviating, and/or decreasing the severity of one or more clinical complication of a condition being treated (e.g., decreasing a risk of telangiectasia in a patient receiving a therapeutically effective amount of an ActRII polypeptide). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., decreasing a risk of telangiectasia in a patient receiving a therapeutically effective amount of an ActRII polypeptide) is achieved by administering one or more ActRII polypeptides of the present disclosure in an “effective amount”. An effective amount of an agent refers to an amount effective, at dosages and for periods of time necessary, to achieve the desired therapeutic or prophylactic result. A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of an ActRII polypeptide, in combination with one or more additional active agents or other supportive therapy for treating or preventing a disease or condition (e.g., decreasing a risk of telangiectasia in a patient receiving a therapeutically effective amount of an ActRII polypeptide). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more ActRII polypeptides of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII polypeptide of the present disclosure with the additional active agent or therapy and/or the desired effect.

### WHO Classification Outline

A pulmonary arterial hypertension condition treated by methods describe herein, can comprise any one or more of the conditions recognized according to the World Health Organization (WHO). See, e.g., Simonneau (2019) Eur Respir J: 53:1801913.

TABLE 1
Clinical Classification of Pulmonary Arterial Hypertension

```
Group 1: Pulmonary arterial hypertension (PAH)
1.1 Idiopathic PAH
1.2 Heritable PAH
  1.2.1 BMPR2
  1.2.2 ALK-1, ENG, SMAD9, CAVI, KCNK3
  1.2.3 Unknown
1.3 Drug and toxin induced PAH
1.4 Associated with:
  1.4.1 Connective tissue disease
  1.4.2 HIV infection
  1.4.3 Portal hypertension
  1.4.4 Congenital heart diseases
  1.4.5 Schistosomiasis
1.5 PAH long-term responders to calcium channel blockers
1.6 PAH with overt features of venous/capillaries (PVOD/PCH)
    involvement
1.7 Persistent PH of the newborn syndrome
```

The clinical purpose of the classification of PAH is to categorize clinical conditions associated with PAH into specific subgroups according to their pathophysiological mechanisms, clinical presentation, hemodynamic characteristics, and treatment strategy. This clinical classification may be updated when new data are available on the above features or when additional clinical entities are considered.

As used herein, the term “pulmonary hemodynamic parameter” refers to any parameter used to describe or evaluate the blood flow through the heart and pulmonary vasculature. Examples of pulmonary hemodynamic parameters include, but are not limited to, mean pulmonary artery pressure (mPAP), diastolic pulmonary artery pressure (dPAP) [also known as pulmonary artery diastolic pressure (PADP)], systolic pulmonary artery pressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], pulmonary vascular resistance (PVR) and cardiac output (CO).

Many of the pulmonary hemodynamic parameters described above are interrelated. For example, PVR is related to mPAP, PCWP and CO according to the following equation:

$$PVR = ( mPAP - PCWP ) / CO [ Woods Units ]$$

The PVR measures the resistance to flow imposed by the pulmonary vasculature without the influence of the left-sided filling pressure. PVR can also be measured according to the following equations:

$$PVR = TPG \times 80 / CO [ \text{unit:} dynes - sec - {cm}^{- 5} ] OR PVR = ( mPAP - PCWP ) \times 80 / CO [ \text{unit:} dynes - sec - {cm}^{- 5} ]$$

In some embodiments, the total peripheral resistance (TPR) can be measured using the following equation:

$$TPR = mPAP / CO .$$

According to some embodiments, a pre-capillary pulmonary arterial contribution to PH may be reflected by an elevated PVR. In some embodiments, the normal PVR is 20-130 dynes-sec-cm⁻⁵ or 0.5-1.1 Wood units. According to some embodiments, an elevated PVR may refer to a PVR above 2 Wood units, above 2.5 Wood units, above 3 Wood units or above 3.5 Wood units.

As yet another example, mPAP is related to dPAP and sPAP according to the following equation: mPAP=(⅔)dPAP+(⅓) sPAP

Furthermore, dPAP and sPAP can be used to calculate the pulse pressure (mmHg) using the following equation: pulse pressure=sPAP−dPAP

Pulse pressure can be used to calculate the pulmonary artery compliance using the following equation: pulmonary artery compliance (ml·mmHg⁻¹)=stroke volume/pulse pressure

In some embodiments, the pulmonary hemodynamic parameters are measured directly, such as during a right heart catheterization. In other embodiments, the pulmonary hemodynamic parameters are estimated and/or evaluated through other techniques such as magnetic resonance imaging (MRI) or echocardiography.

Exemplary pulmonary hemodynamic parameters include mPAP, PAWP, and PVR. The one or more pulmonary hemodynamic parameters may be measured by any appropriate procedures, such as by utilizing a right heart catheterization or echocardiography. Various hemodynamic characteristics of PH and PAH are shown in Table 2.

TABLE 2
Hemodynamic Characteristics of Pulmonary
Hypertension (PH) and PAH

| | Hemodynamic Characteristics |
|---|---|
| Pulmonary | mPAP >20 mmHg |
| Hypertension | |
| Pulmonary | mPAP >20 mmHg |
| arterial | |
| hypertension | PAWP ≤15 mmHg |
| | PVR ≥3 Wood units |

The clinical classification or hemodynamic characteristics of PAH described herein and the associated diagnostic parameters may be updated or varied based on the availability of new or existing sources of data or when additional clinical entities are considered.

### Characteristics of PAH

Pulmonary arterial hypertension (WHO Group 1 PH) is a serious, progressive and life-threatening disease of the pulmonary vasculature, characterized by profound vasoconstriction and an abnormal proliferation of smooth muscle cells in the walls of the pulmonary arteries. Severe constriction of the blood vessels in the lungs leads to very high pulmonary artery pressures. These high pressures make it difficult for the heart to pump blood through the lungs to be oxygenated. Patients with PAH suffer from extreme shortness of breath as the heart struggles to pump against these high pressures. Patients with PAH typically develop significant increases in PVR and sustained elevations in mPAP, which ultimately lead to right ventricular failure and death. Patients diagnosed with PAH have a poor prognosis and equally compromised quality of life, with a mean life expectancy of 2 to 5 years from the time of diagnosis if untreated.

A variety of factors contribute to the pathogenesis of pulmonary hypertension including proliferation of pulmonary cells which can contribute to vascular remodeling (i.e., hyperplasia). For example, pulmonary vascular remodeling occurs primarily by proliferation of arterial endothelial cells and smooth muscle cells of patients with pulmonary hypertension. Overexpression of various cytokines is believed to promote pulmonary hypertension. Further, it has been found that pulmonary hypertension may rise from the hyperproliferation of pulmonary arterial smooth cells and pulmonary endothelial cells. Still further, advanced PAH may be characterized by muscularization of distal pulmonary arterioles, concentric intimal thickening, and obstruction of the vascular lumen by proliferating endothelial cells. Pietra et al., J. Am. Coll. Cardiol., 43:255-325 (2004).

PAH can be diagnosed based on a mean pulmonary artery pressure of above 25 mmHg (or above 20 mmHg under updated guidelines) at rest, with a normal pulmonary artery capillary wedge pressure. PAH can lead to shortness of breath, dizziness, fainting, and other symptoms, all of which are exacerbated by exertion. PAH can be a severe disease with a markedly decreased exercise tolerance and heart failure. Two major types of PAH include idiopathic PAH (e.g., PAH in which no predisposing factor is identified) and heritable PAH (e.g., PAH associated with a mutation in BMPR2, ALK1, ENG, SMAD9, CAV1, KCNK3, or EIF2AK4). In 70% of familial PAH cases, mutations are located in the BMPR2 gene. Risk factors for the development of PAH include family history of PAH, drug and toxin use (e.g., methamphetamine or cocaine use), infection (e.g., HIV infection or schistosomiasis), cirrhosis of the liver, congenital heart abnormalities, portal hypertension, pulmonary veno-occlusive disease, pulmonary capillary hemangiomatosis, or connective tissue/autoimmune disorders (e.g., scleroderma or lupus). PAH may be associated with long term responders to calcium channel blockers, overt features of venous/capillaries (PVOD/PCH) involvement, and persistent PH of the newborn syndrome.

### Diagnosis of PAH

The diagnosis of PAH, including functional group, can be determined based on symptoms and physical examination using a review of a comprehensive set of parameters to determine if the hemodynamic and other criteria are met. Some of the criteria which may considered include the patient's clinical presentation (e.g., shortness of breath, fatigue, weakness, angina, syncope, dry-couch, exercise-induced nausea and vomiting), electrocardiogram (ECG) results, chest radiograph results, pulmonary function tests, arterial blood gases, echocardiography results, ventilation/perfusion lung scan results, high-resolution computed tomography results, contrast-enhanced computed tomography results, pulmonary angiography results, cardiac magnetic resonance imaging, blood tests (e.g., biomarkers such as BNP or NT-proBNP), immunology, abdominal ultrasound scan, right heart catherization (RHC), vasoreactivity, and genetic testing. See, e.g., Galie N., et al Euro Heart J. (2016) 37, 67-119.

In some embodiments, a biomarker may be used to aid in the diagnosis of PAH. For instance, in some embodiments, the biomarker is a marker of vascular dysfunction (e.g., asymmetric dimethylarginine (ADMA), endothelin-1, angiopoeitins, or von Willebrand factor). In some embodiments, the biomarker is a marker of inflammation (C-reactive protein, interleukin 6, chemokines). In some embodiments, the biomarker is a marker of myocardial stress [e.g., (atrial natriuretic peptide, brain natriuretic peptide (BNP)/NT-proBNP, or troponins]. In some embodiments, the biomarker is a marker of low CO and/or tissue hypoxia (e.g., pCO2, uric acid, growth differentiation factor 15 (GDF15), or osteopontin). In some embodiments, the biomarker is a marker of secondary organ damage (e.g., creatinine or bilirubin). See, e.g., Galie N., et al Euro Heart J. (2016) 37, 67-119.

### Measurements of PH

In certain aspects, the disclosure relates to methods of decreasing a risk of telangiectasia in a patient receiving a therapeutically effective amount of an ActRII polypeptide (e.g., an amino acid sequence that is at least 90% identical to an amino acid sequence corresponding to residues 30-110 of SEQ ID NO: 1) for the treatment and/or progression of pulmonary arterial hypertension (PAH). In some embodiments, the method relates to treating PAH patients that have idiopathic PAH. In some embodiments, the method relates to treating PAH patients that have heritable PAH (e.g., PAH due to one or more mutations within BMPR2, ALK-1, ENG, SMAD9, CAV1, and KCNK3). In some embodiments, the method relates to treating PAH patients that have heritable PAH due to an unknown mutation. In some embodiments, the method relates to treating PAH patients that have drug or toxin induced PAH. In some embodiments, the method relates to treating PAH patients that have PAH associated with connective tissue disease. In some embodiments, the method relates to treating PAH patients that have PAH associated with HIV infection. In some embodiments, the method relates to treating PAH patients that have PAH associated with portal hypertension. In some embodiments, the method relates to treating PAH patients that have PAH associated with schistosomiasis. In some embodiments, the method relates to treating PAH patients classified as long-term responders to calcium channel blockers. In some embodiments, the method relates to treating PAH patients with overt features of venous/capillaries (PVOD/PCH) involvement. In some embodiments, the method relates to treating PAH patients that have persistent pulmonary hypertension (PH) of the newborn syndrome. In some embodiments, the method relates to treating PAH patients that have PAH associated with simple, congenital systemic-to-pulmonary shunts at least 1 year following shunt repair.

### Functional Classes

PAH at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Functional Class I: pulmonary hypertension without resulting limitation of physical activity; ordinary physical activity does not cause undue dyspnea or fatigue, chest pain or near syncope; Functional Class II: pulmonary hypertension resulting in slight limitation of physical activity; patient comfortable at rest; ordinary physical activity causes undue dyspnea or fatigue, chest pain or near syncope; Functional Class III: pulmonary hypertension resulting in marked limitation of physical activity; patient comfortable at rest; less than ordinary activity causes undue dyspnea or fatigue, chest pain or near syncope; Functional Class IV: pulmonary hypertension resulting in inability to carry out any physical activity without symptoms; patient manifests signs of right-heart failure; dyspnea and/or fatigue may be present even at rest; discomfort is increased by any physical activity.

### Known Treatments for PAH

There is no known cure for PAH; current methods of treatment focus on prolonging patient lifespan and enhancing patient quality of life. This is usually associated with good exercise capacity, good right ventricle function, and a low mortality risk (e.g., bring and/or keeping the patient in WHO Functional Class I or Functional Class II). Current methods of treatment of PAH may include administration of: vasodilators such as prostacyclin, epoprostenol, and sildenafil; endothelin receptor antagonists such as bosentan; calcium channel blockers such as amlodipine, diltiazem, and nifedipine; anticoagulants such as warfarin; and diuretics. Treatment of PAH has also been carried out using oxygen therapy, atrial septostomy, pulmonary thromboendarterectomy, and lung and/or heart transplantation. Each of these methods, however, suffers from one or multiple drawbacks which may include lack of effectiveness, serious side effects, low patient compliance, and high cost. In certain aspects, the method relates to decreasing a risk of telangiectasia in a patient receiving a therapeutically effective amount of an ActRII polypeptide (e.g., an amino acid sequence that is at least 90% identical to an amino acid sequence corresponding to residues 30-110 of SEQ ID NO: 1) in combination with one or more additional active agents and/or supportive therapies for treating PAH (e.g., vasodilators such as prostacyclin, epoprostenol, and sildenafil; endothelin receptor antagonists such as bosentan; calcium channel blockers such as amlodipine, diltiazem, and nifedipine; anticoagulants such as warfarin; diuretics; oxygen therapy; atrial septostomy; pulmonary thromboendarterectomy; and lung and/or heart transplantation); bardoxolone methyl or a derivative thereof; oleanolic acid or derivative thereof.

### Dosing Regimen for Decreasing Risk of Telangiectasia

The probability of a patient having telangiectasia may be higher during initial treatment with an ActRII polypeptide. In certain embodiments, a dosing regimen can be used to prevent, ameliorate, or decrease the symptoms of telangiectasia. In some embodiments, ActRII polypeptides of the disclosure are administered using a dosing regimen. In some embodiments, the method comprises administering a dosing regimen of a therapeutically effective amount of an ActRII polypeptide as disclosed herein to a patient, comprising a first dose of between 0.1 mg/kg and 1.0 mg/kg of said polypeptide for a first period of time, and a second dose of between 0.1 mg/kg and 1.0 mg/kg of said polypeptide subsequently administered for a second period of time. In some embodiments, the method comprises administering a dosing regimen of therapeutically effective amount of an ActRII polypeptide as disclosed herein to a patient, comprising a first dose of between 0.1 mg/kg and 1.0 mg/kg of said polypeptide for a first period of time, a second dose of between 0.1 mg/kg and 1.0 mg/kg of said polypeptide administered for a second period of time, and a third dose of between 0.1 mg/kg and 1.0 mg/kg of said polypeptide subsequently administered for a third period of time. In some embodiments, the method comprises administering a dosing regimen of therapeutically effective amount of an ActRII polypeptide as disclosed herein to a patient, comprising a first dose of between 0.1 mg/kg and 1.0 mg/kg of said polypeptide for a first period of time, a second dose of between 0.1 mg/kg and 1.0 mg/kg of said polypeptide administered for a second period of time, and a third dose wherein the ActRII polypeptide treatment is withheld for a third period of time. In some embodiments, the first dose of ActRII polypeptide is administered to a patient in an amount from about 0.2 mg/kg to about 0.4 mg/kg. In some embodiments, the first dose of ActRII polypeptide is administered to a patient at a dose of 0.3 mg/kg. In some embodiments, the second dose of ActRII polypeptide is administered to a patient in an amount from about 0.5 mg/kg to about 0.8 mg/kg. In some embodiments, the second dose of ActRII polypeptide is administered to a patient at a dose of 0.7 mg/kg. In some embodiments, the third dose of ActRII polypeptide is administered to a patient in an amount from about 0.2 mg/kg to about 0.4 mg/kg. In some embodiments, the third dose of ActRII polypeptide is administered to a patient at a dose of 0.3 mg/kg. In some embodiments, the third dose comprises withholding treatment of the ActRII polypeptide for a period of time. In some embodiments, the ActRII polypeptide treatment is withheld for a period of at least 2-6 weeks. In some embodiments, the ActRII polypeptide treatment is withheld for a period of at least 3 weeks. In some embodiments, the ActRII polypeptide treatment is withheld for a period of at least 6 weeks. In some embodiments, the ActRII polypeptide treatment is withheld for a period of at least 9 weeks. In some embodiments, the ActRII polypeptide treatment is resumed following the period during which the ActRII polypeptide treatment is withheld.

In some embodiments, the dosing regimen comprises administering a first dose of ActRII polypeptide to a patient in an amount of 0.3 mg/kg followed by administration of a second dose of ActRII polypeptide to the patient in an amount of 0.7 mg/kg. In some embodiments, the dosing regimen comprises administering a first dose of ActRII polypeptide to a patient in an amount of 0.3 mg/kg, administering a second dose of ActRII polypeptide to the patient in an amount of 0.7 mg/kg, and administering a third dose of ActRII polypeptide to the patient in an amount of 0.3 mg/kg. In some embodiments, the dosing regimen comprises administering a first dose of ActRII polypeptide to a patient in an amount of 0.3 mg/kg, administering a second dose of ActRII polypeptide to the patient in an amount of 0.7 mg/kg, and administering a third dose of ActRII polypeptide to the patient wherein the third dose comprises withholding treatment of the ActRII polypeptide for a period of at least 3 weeks. In some embodiments, the dosing regimen comprises administering a first dose of ActRII polypeptide to a patient in an amount of 0.3 mg/kg and administering a second dose of ActRII polypeptide to the patient wherein the second dose comprises withholding treatment of the ActRII polypeptide for a period of at least 3 weeks. In some embodiments, the dosing regimen comprises administering a first dose of ActRII polypeptide to a patient in an amount of 0.3 mg/kg, administering a second dose of ActRII polypeptide to the patient in an amount of 0.7 mg/kg, administering a third dose of ActRII polypeptide to the patient wherein the third dose comprises withholding treatment of the ActRII polypeptide for a period of at least 3 weeks, and administering a fourth dose of ActRII polypeptide to the patient in an amount of 0.3 mg/kg. In some embodiments, the second dose exceeds the first dose. In some embodiments, the first dose exceeds the second dose. In some embodiments, the third dose exceeds the second dose. In some embodiments, the second dose exceeds the third dose. In some embodiments, the third dose comprises administering a dose of ActRII polypeptide to the patient in an amount of 0.3 mg/kg. In some embodiments, the third dose comprises withholding treatment of the ActRII polypeptide for a period of at least 2-6 weeks. In some embodiments, the the second dose comprises withholding treatment of the ActRII polypeptide for a period of at least 2-6 weeks. In some embodiments, the third dose comprises withholding treatment of the ActRII polypeptide for a period of at least 3 weeks. In some embodiments, the second dose comprises withholding treatment of the ActRII polypeptide for a period of at least 3 weeks. In some embodiments, the first period of time is at least 3 weeks. In some embodiments, the second period of time is at least 3 weeks. In some embodiments, the third period of time is at least 3 weeks. In some embodiments, the second period of time is at least 21 weeks. In some embodiments, the second period of time is at least 45 weeks. In some embodiments, the second period of time exceeds the first period of time. In some embodiments, the third period of time exceeds the first period of time. In some embodiments, the third period of time exceeds the second period of time. In some embodiments, the first dose is administered to the patient for at least 3 weeks. In some embodiments, the second dose is administered for at least 21 weeks. In some embodiments, the second dose is administered for at least 45 weeks.

In some embodiments, the change in dosing between the first dose and the second dose is determined by the attending physician considering various factors (e.g., symptoms and/or risk factors of telangiectasia). In some embodiments, the change in dosing between the second dose and the third dose is determined by the attending physician considering various factors (e.g., symptoms and/or risk factors of telangiectasia). In some embodiments, the change in dosing between the third dose and the fourth dose is determined by the attending physician considering various factors (e.g., symptoms and/or risk factors of telangiectasia).

In some embodiments, the various factors include, but are not limited to, the patient's risk factors for developing telangiectasia. In some embodiments, risk factors for developing telangiectasia are selected from the group consisting of low BMP9 levels, low BMP10 levels, low VEGF levels, hereditary hemorrhagic telangiectasia (HHT), and connective tissue disease (CTD). In some embodiments, the patient comprises one or more of these risk factors prior to treatment with an ActRII polypeptide. In some embodiments, the patient comprises one or more of these risk factors during treatment with an ActRII polypeptide. In some embodiments, the risk factor for developing telangiectasia is the patient comprising one or more mutations associated with hereditary hemorrhagic telangiectasia (HHT) and/or connective tissue disease (CTD). In some embodiments, the one or more mutations associated with HHT are mutations in a gene selected from the group consisting of ENG, ACVRL1, EPHB4, SMAD4, GDF2, BMPR9, and RASA1. In some embodiments, the one or more mutations associated with CTD are mutations in a gene selected from the group consisting of ABCC6, ACTA2, ADAMTS2, ADAMTS10, ADAMTSL2, ALDH18A1, ATP6V0A2, ATP7A, B3GALT6, B4GALT7, BGN, CIR, CIS, CBS, CHST14, COL1A1, COL1A2, COL2A1, COL3A1, COL5A1, COL5A2, COL9A1, COL9A2, COL9A3, COL11A1, COL11A2, DSE, EFEMP2 (FBLN4), ELN, FBLN5, FBN1, FBN2, FKBP14, FLCN, FLNA, FOXE3, GORAB, LOX, LTBP4, MED12, MFAP5, MYH11, MYLK, NOTCH1, NOTCH2, PKD1, PKD2, PLOD1, PRDM5, PRKG1, PTDSS1, PYCR1, RIN2, SK1, SLC2A10, SLC39A13, SMAD2, SMAD3, SMAD4, SMAD6, TAB2, TGFB2, TGFB3, TGFBR1, TGFBR2, TNXB, and ZNF469. In some embodiments, the patient's dose of an ActRII polypeptide as disclosed herein will be maintained (e.g., maintained at 0.3 mg/kg or 0.7 mg/kg) if one or more of the patient's symptoms or risk factors before or during treatment is abnormal.

In some embodiments, the dosing regimen prevents, ameliorates, or decreases adverse effects of the ActRII polypeptide. In some embodiments, administration of an ActRII polypeptide in accordance with the dosage regimen as provided herein results in decreased risk of telangiectasia. In some embodiments, administration of an ActRII polypeptide in accordance with the dosage regimen as provided herein decreases the risk of telangiectasia during the second period of time. In some embodiments, administration of an ActRII polypeptide in accordance with the dosage regimen as provided herein decreases the risk of telangiectasia during the third period of time. In some embodiments, administration of an ActRII polypeptide in accordance with the dosage regimen as provided herein decreases the risk of telangiectasia during the fourth period of time. In some embodiments, administration of an ActRII polypeptide in accordance with the dosage regimen as provided herein decreases the risk of telangiectasia after the first 21 weeks of treatment. In some embodiments, administration of an ActRII polypeptide in accordance with the dosage regimen as provided herein decreases the risk of telangiectasia after the first 45 weeks of treatment.

### Symptoms and Risk Factors for Telangiectasia

Telangiectasia are also known as spider veins, hyphen webs, thread veins, sunburst veins, stellate veins and venous flares. Telangiectasia comprise widened venules (tiny blood vessels) which result in threadlike red lines or patterns on the skin. These patterns, or telangiectases, are thought to be caused by the release or activation of vasoactive substances under a multitude of conditions. Symptoms of telangiectasia include pain, itching, threadlike red marks on the skin, mucocutaneous telangiectases, gastrointestinal bleeding, lesions on the skin, epistaxis, gingival bleeding, arteriovenous malformations, internal telangiectases, and red spots on the skin.

While telangiectases are often benign, they may be caused by a serious illness such as hereditary hemorrhagic telangiectasia (HHT). In patients with HHT, telangiectases may appear in vital organs, such as the liver. The rupture of these telangiectases may result in life-threatening hemorrhaging.

In certain aspects, the disclosure relates to methods of decreasing a risk of telangiectasia in a patient receiving a therapeutically effective amount of an ActRII polypeptide (e.g., an amino acid sequence that is at least 90% identical to an amino acid sequence corresponding to residues 30-110 of SEQ ID NO: 1), wherein the method comprises administering the ActRII polypeptide on a dosing regimen if the patient shows symptoms or comprises risk factors for developing telangiectasia, thereby decreasing the risk of telangiectasia. In some embodiments, the symptoms of telangiectasia are selected from the group consisting of pain, itching, threadlike red marks on the skin, mucocutaneous telangiectases, gastrointestinal bleeding, lesions on the skin, epistaxis, gingival bleeding, arteriovenous malformations, internal telangiectases, and red spots on the skin. In some embodiments, the symptom of telangiectasia is lesions on the skin. In some embodiments, the symptom of telangiectasia is gingival bleeding. In some embodiments, the symptom of telangiectasia is epistaxis. In some embodiments, the symptom of telangiectasia is arteriovenous malformations. In some embodiments, the symptom of telangiectasia is internal telangiectases. In some embodiments, the arteriovenous malformations or internal telangiectases occur in internal organs (e.g., brain, liver, lungs, spleen, urinary tract, and spine). In some embodiments, the risk factors for developing telangiectasia are selected from the group consisting of low BMP9 levels; low BMP10 levels; low VEGF levels; hereditary hemorrhagic telangiectasia (HHT); and connective tissue disease (CTD).

In certain embodiments, the present disclosure provides methods for managing a patient that has been treated with, or is a candidate to be treated with, one or more one or more ActRII polypeptides of the disclosure (e.g., an amino acid sequence that is at least 90% identical to an amino acid sequence corresponding to residues 30-110 of SEQ ID NO: 1) by measuring one or more symptoms or risk factors of telangiectasia in the patient. The one or more symptoms and/or risk factors of telangiectasia may be used to evaluate appropriate dosing for a patient who is a candidate to be treated with one or more ActRII polypeptides of the present disclosure, to monitor the symptoms and/or risk factors of telangiectasia during treatment, to evaluate whether to adjust the dosage during treatment with one or more ActRII polypeptides of the disclosure, and/or to evaluate an appropriate maintenance dose of one or more ActRII polypeptides of the disclosure. If one or more of the symptoms and/or risk factors of telangiectasia are abnormal, dosing with one or more ActRII polypeptides may be reduced, delayed or terminated.

In one embodiment, if one or more symptoms and/or risk factors of telangiectasia are abnormal in a patient who is a candidate to be treated with one or more ActRII polypeptides, then onset of administration of the one or more ActRII polypeptides of the disclosure may be delayed until the symptoms and/or risk factors of telangiectasia have returned to a normal or acceptable level either naturally or via therapeutic intervention.

In certain embodiments, if one or more symptoms and/or risk factors of telangiectasia are abnormal in a patient who is a candidate to be treated with one or more ActRII polypeptides, then the onset of administration may not be delayed. However, the dosage amount or frequency of dosing of the one or more ActRII polypeptides of the disclosure may be set at an amount that would reduce the risk of telangiectasia arising upon administration of the one or more ActRII polypeptides of the disclosure. Alternatively, a therapeutic regimen may be developed for the patient that combines one or more ActRII polypeptides with a therapeutic agent that addresses the risk of telangiectasia.

### Severity of Telangiectasia in a Patient

Telangiectasia and its severity level can be described and measured using various classifications. For instance, the Clinical-Etiology-Anatomy-Pathophysiology (CEAP) classification for chronic venous disorders grades venous conditions in order of increasing severity. CEAP categorizes telangiectasia in the least severe category (C1) which describes telangiectasia as a confluence of dilated intradermal venules less than 1 mm in calibre. See, e.g., Table 3 below.

TABLE 3
CEAP Clinical Classification for Chronic Venous Disease
Clinical Classification

| | |
|---|---|
| CO | No visible or palpable signs of disease |
| C1 | Telangiectasias or reticular veins |
| C2 | Varicose Veins |
| C3 | Edema |
| C4a | Pigmentation or eczema |
| C4b | Lipodermatosclerosis or atrophic blanche |
| C5 | Healed venous ulcer |
| C6 | Active venous ulcer |
| S | Symptomatic, including ache, pain, tightness, skin irritation, |
| | heaviness, muscle cramps, and other complains attributable to |
| | venous dysfunction |
| A | Asymptomatic |

Telangiectasia can be further defined into specific grades of severity. For instance, telangiectasia can be graded into three grades according to the degree and extent of tortuosity and prominence of the veins. See, e.g., Ruckley C V, et al. European Journal of Vascular and Endovascular Surgery. 2008; 36 (6): 719-724.

In certain aspects, the disclosure relates to methods of decreasing a risk of telangiectasia in a patient receiving a therapeutically effective amount of an ActRII polypeptide (e.g., an amino acid sequence that is at least 90% identical to an amino acid sequence corresponding to residues 30-110 of SEQ ID NO: 1), wherein the patient has C1 telangiectasia as classified by the Clinical-Etiology-Anatomy-Pathophysiology (CEAP). In some embodiments, method improves the telangiectasia from C1 to CO as classified by the CEAP.

In certain aspects, the disclosure relates to methods of decreasing a risk of telangiectasia in a patient receiving a therapeutically effective amount of an ActRII polypeptide (e.g., an amino acid sequence that is at least 90% identical to an amino acid sequence corresponding to residues 30-110 of SEQ ID NO: 1), wherein the patient has grade 1, grade 2, or grade 3 telangiectasia. In some embodiments, the patient has grade 1 telangiectasia. In some embodiments, the patient has grade 2 telangiectasia. In some embodiments, the patient has grade 3 telangiectasia. In some embodiments, the risk of telangiectasia is decreased by alleviating or improving the severity telangiectasia. In some embodiments, the risk of telangiectasia is decreased by improving the grade of telangiectasia. In some embodiments, the method improves the telangiectasia from grade 2 to grade 1. In some embodiments, the method improves the telangiectasia from grade 3 to grade 2. In some embodiments, the method improves the telangiectasia from grade 3 to grade 1. In some embodiments, the risk of telangiectasia is decreased by preventing the progression of the telangiectasia. In some embodiments, the method prevents the progression of the telangiectasia from grade 1 to grade 2. In some embodiments, the method prevents the progression of the telangiectasia from grade 2 to grade 3.

In certain aspects, the disclosure relates to methods of decreasing a risk of telangiectasia in a patient receiving a therapeutically effective amount of an ActRII polypeptide (e.g., an amino acid sequence that is at least 90% identical to an amino acid sequence corresponding to residues 30-110 of SEQ ID NO: 1), wherein the method comprises administering the ActRII polypeptide on a dosing regimen comprising (i) administering a first dose of ActRII polypeptide to a patient in an amount of 0.3 mg/kg; and (ii) administering a second dose of ActRII polypeptide to the patient in an amount of 0.7 mg/kg; wherein a third dose is initiated if the patient shows symptoms or comprises risk factors for developing telangiectasia, thereby decreasing the risk of telangiectasia. In some embodiments, the third dose comprises withholding treatment of the ActRII polypeptide until the telangiectasia improves to ≤ grade 1. In some embodiments, the third dose comprises withholding treatment of the ActRII polypeptide until the telangiectasia improves to ≤ grade 2. In some embodiments, the third dose comprises withholding treatment of the ActRII polypeptide until the telangiectasia improves to ≤ grade 3.

### 5. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII polypeptides which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., ActRII polypeptides) to a target tissue site, providing a structure for the developing tissue and optimally capable of being resorbed into the body. For example, the matrix may provide slow release of the ActRII polypeptide. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII polypeptides). The various factors include, but are not limited to, the patient's age, sex, and diet, the severity disease, time of administration, and other clinical factors. Optionally, the dosage may vary with the type of matrix used in the reconstitution and the types of compounds in the composition. In some embodiments, a patient's hematologic parameters can be monitored by periodic assessments in order to determine if they have higher than normal red blood cell levels and/or hemoglobin levels (e.g., hemoglobin levels >16.0 g/dL or hemoglobin levels >18.0 g/dL). In some embodiments, a patient having higher than normal red blood cell levels and/or hemoglobin levels may receive a delayed or reduced dose until the levels have returned to a normal or acceptable level.

In some embodiments, ActRII polypeptides of the disclosure are administered at a dosing range of 0.1 mg/kg to 2.0 mg/kg. In some embodiments, ActRII polypeptides of the disclosure are administered at 0.1 mg/kg. In some embodiments, ActRII polypeptides of the disclosure are administered at 0.2 mg/kg. In some embodiments, ActRII polypeptides of the disclosure are administered at 0.3 mg/kg. In some embodiments, ActRII polypeptides of the disclosure are administered at 0.4 mg/kg. In some embodiments, ActRII polypeptides of the disclosure are administered at 0.5 mg/kg. In some embodiments, ActRII polypeptides of the disclosure are administered at 0.6 mg/kg. In some embodiments, ActRII polypeptides of the disclosure are administered at 0.7 mg/kg. In some embodiments, ActRII polypeptides of the disclosure are administered at 0.8 mg/kg. In some embodiments, ActRII polypeptides of the disclosure are administered at 0.9 mg/kg. In some embodiments, ActRII polypeptides of the disclosure are administered at 1.0 mg/kg. In some embodiments, ActRII polypeptides of the disclosure are administered at 1.1 mg/kg. In some embodiments, ActRII polypeptides of the disclosure are administered at 1.2 mg/kg. In some embodiments, ActRII polypeptides of the disclosure are administered at 1.3 mg/kg. In some embodiments, ActRII polypeptides of the disclosure are administered at 1.4 mg/kg. In some embodiments, ActRII polypeptides of the disclosure are administered at 1.5 mg/kg. In some embodiments, ActRII polypeptides of the disclosure are administered at 1.6 mg/kg. In some embodiments, ActRII polypeptides of the disclosure are administered at 1.7 mg/kg. In some embodiments, ActRII polypeptides of the disclosure are administered at 1.8 mg/kg. In some embodiments, ActRII polypeptides of the disclosure are administered at 1.9 mg/kg. In some embodiments, ActRII polypeptides of the disclosure are administered at 2.0 mg/kg.

In certain embodiments, ActRII polypeptides of the disclosure are administered once a day. In certain embodiments, ActRII polypeptides of the disclosure are administered twice a day. In certain embodiments, ActRII polypeptides of the disclosure are administered once a week. In certain embodiments, ActRII polypeptides of the disclosure are administered twice a week. In certain embodiments, ActRII polypeptides of the disclosure are administered three times a week. In certain embodiments, ActRII polypeptides of the disclosure are administered every two weeks. In certain embodiments, ActRII polypeptides of the disclosure are administered every three weeks. In certain embodiments, ActRII polypeptides of the disclosure are administered every four weeks. In certain embodiments, ActRII polypeptides of the disclosure are administered every month.

In certain embodiments, the present invention also provides gene therapy for the in vivo production of ActRII polypeptides. Such therapy would achieve its therapeutic effect by introduction of the ActRII polypeptide polynucleotide sequences into cells or tissues having the disorders as listed above. Delivery of ActRII polypeptide polynucleotide sequences can be achieved using a recombinant expression vector such as a chimeric virus or a colloidal dispersion system. Preferred for therapeutic delivery of ActRII polypeptide polynucleotide sequences is the use of targeted liposomes.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing the ActRII polypeptide. In a preferred embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII polypeptide polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### 6. Exemplification

The disclosure above will be more readily understood by reference to the following examples, which are included merely for purposes of illustration of certain embodiments of the present invention, and are not intended to limiting.

### Example 1: ActRIIA-Fc Fusion Proteins

A soluble ActRIIA fusion protein was constructed that has the extracellular domain of human ActRIIa fused to a human or mouse Fc domain with a minimal linker in between. The constructs are referred to as ActRIIA-hFc and ActRIIA-mFc, respectively.

ActRILA-hFc is shown below as purified from CHO cell lines (SEQ ID NO: 23):

ILGRSETQECLFFNANWEKDRTNQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG
K

An additional ActRIIA-hFc lacking the C-terminal lysine is shown below as purified from CHO cell lines (SEQ ID NO: 40):

ILGRSETQECLFFNANWEKDRINQTGVEPCYGDKDKRRHCFATWKNISG
SIEIVKQGCWLDDINCYDRTDCVEKKDSPEVYFCCCEGNMCNEKFSYFP
EMEVTQPTSNPVTPKPPTGGGTHTCPPCPAPELLGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPVPIEKTISKAKGQPREPQV
YTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG

The ActRILA-hFc and ActRIIA-mFc proteins were expressed in CHO cell lines. Three different leader sequences were considered:

(i) Honey bee mellitin (HBML):
(SEQ ID NO: 24)
MKFLVNVALVEMVVYISYIYA
(ii) Tissue plasminogen activator (TPA):
(SEQ ID NO: 25)
MDAMKRGLCCVLLLCGAVEVSP
(iii) Native:
(SEQ ID NO: 26)
MGAAAKLAFAVFLISCSSGA

The selected form employs the TPA leader and has the following unprocessed amino acid sequence:

(SEQ ID NO: 27)
MDAMKRGLCCVLLLCGAVEVSPGAAILGRSETQECLFFNANWEKDRINQ
TGVEPCYGDKDKRRHCFATWKNISGSIEIVKQGCWLDDINCYDRTDCVE
KKDSPEVYFCCCEGNMCNEKFSYFPEMEVTQPTSNPVTPKPPTGGGTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPVPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK

This polypeptide is encoded by the following nucleic acid sequence:

(SEQ ID NO: 28)
ATGGATGCAATGAAGAGAGGGCTCTGCTGTGTGCTGCTGCTGTGTGGAG
CAGTCTTCGTTTCGCCCGGCGCCGCTATACTTGGTAGATCAGAAACTCA
GGAGTGTCTTTTTTTAATGCTAATTGGGAAAAAGACAGAACCAATCAAA
CTGGTGTTGAACCGTGTTATGGTGACAAAGATAAACGGCGGCATTGTTT
TGCTACCTGGAAGAATATTTCTGGTTCCATTGAATAGTGAAACAAGGTT
GTTGGCTGGATGATATCAACTGCTATGACAGGACTGATTGTGTAGAAAA
AAAAGACAGCCCTGAAGTATATTTCTGTTGCTGTGAGGGCAATATGTGT
AATGAAAAGTTTTCTTATTTTCCGGAGATGGAAGTCACACAGCCCACTT
CAAATCCAGTTACACCTAAGCCACCCACCGGTGGTGGAACTCACACATG
CCCACCGTGCCCAGCACCTGAACTCCTGGGGGGACCGTCAGTCTTCCTC
TTCCCCCCAAAACCCAAGGACACCCTCATGATCTCCCGGACCCCTGAGG
TCACATGCGTGGTGGTGGACGTGAGCCACGAAGACCCTGAGGTCAAGTT
CAACTGGTACGTGGACGGCGTGGAGGTGCATAATGCCAAGACAAAGCCG
CGGGAGGAGCAGTACAACAGCACGTACCGTGTGGTCAGCGTCCTCACCG
TCCTGCACCAGGACTGGCTGAATGGCAAGGAGTACAAGTGCAAGGTCTC
CAACAAAGCCCTCCCAGTCCCCATCGAGAAAACCATCTCCAAAGCCAAA
GGGCAGCCCCGAGAACCACAGGTGTACACCCTGCCCCCATCCCGGGAGG
AGATGACCAAGAACCAGGTCAGCCTGACCTGCCTGGTCAAAGGCTTCTA
TCCCAGCGACATCGCCGTGGAGTGGGAGAGCAATGGGCAGCCGGAGAAC
AACTACAAGACCACGCCTCCCGTGCTGGACTCCGACGGCTCCTTCTTCC
TCTATAGCAAGCTCACCGTGGACAAGAGCAGGTGGCAGCAGGGGAACGT
CTTCTCATGCTCCGTGATGCATGAGGCTCTGCACAACCACTACACGCAG
AAGAGCCTCTCCCTGTCTCCGGGTAAATGAGAATTC

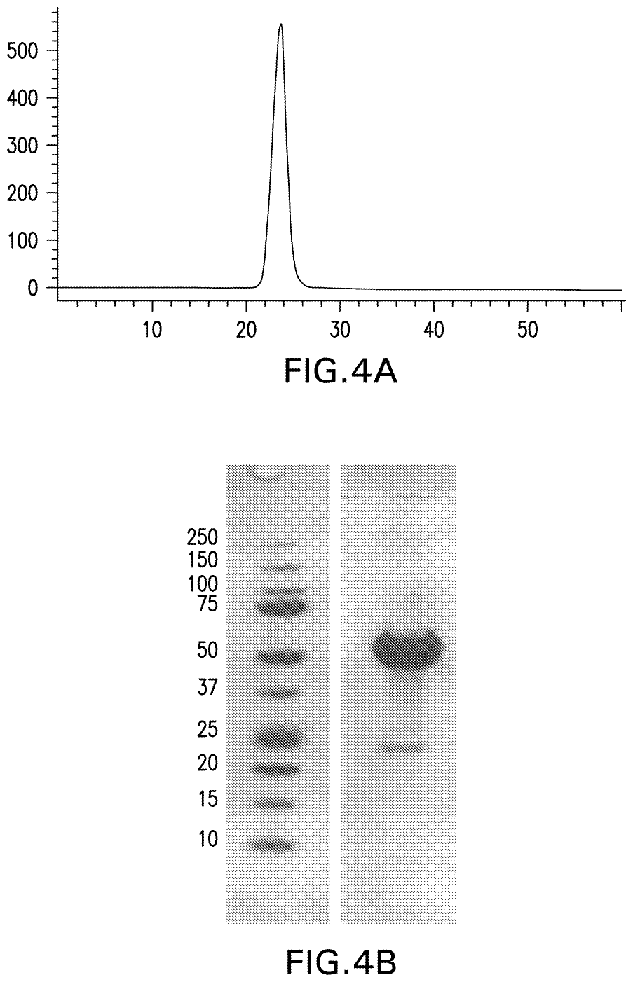

Both ActRIIA-hFc and ActRIIA-mFc were remarkably amenable to recombinant expression. As shown in FIGS. 4A and 4B, the protein was purified as a single, well-defined peak of protein. N-terminal sequencing revealed a single sequence of—ILGRSETQE (SEQ ID NO: 29). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

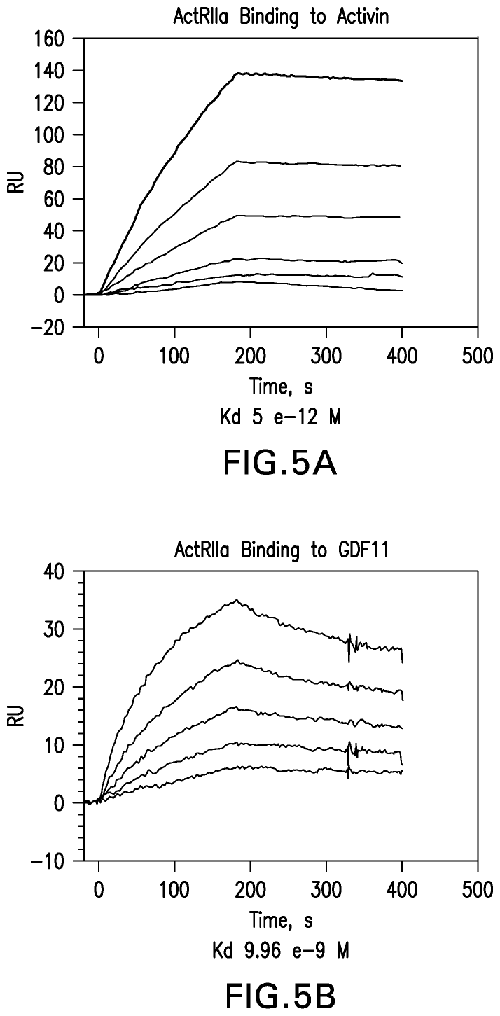

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIG. 5A and FIG. 5B. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In 10 cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 ori/enhancer, CMV promoter), using a tissue plasminogen leader sequence of SEQ ID NO: 25. The protein, purified as described above in Example 1, had a sequence of SEQ ID NO: 23. The Fc portion is a human IgG1 Fc sequence, as shown in SEQ ID NO: 23. Protein analysis reveals that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279 (51): 53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see, e.g., pp. 55-58), incorporated herein by reference in its entirety. An alternative construct may have a deletion of the C-terminal tail (the final 15 amino acids of the extracellular domain of ActRIIA). The sequence for such a construct is presented below (Fc portion underlined) (SEQ ID NO: 30):

ILGRSETQECLFFNANWEKDRINQTGVEPCYGDKDKRRHCFATWKNISG
SIEIVKQGCWLDDINCYDRTDCVEKKDSPEVYFCCCEGNMCNEKESYFP
EMTGGGTHTCPPCPAPELLGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPVPIEKTISKAKGQPREPQVYTLPPSREEMTKNQV
SLTCLVKGFYPSDIAVEWESNGQPENNYKTTPPVLDSDGSFFLYSKLTV
DKSRWQQGNVFSCSVMHEALHNHYTQKSLSLSPGK

1-64. (canceled)

65. A method of reducing a risk of telangiectasia when treating a patient with an ActRIIA fusion protein, the method comprising administering the ActRII fusion protein on a dosing regimen comprising:
(i) administering one or more doses of the ActRII fusion protein to a patient in an amount of 0.3 mg/kg once every 3 weeks for a period of 24 weeks; and
(ii) if the patient shows one or more symptoms or risk factors for developing telangiectasia administering one or more doses of the ActRII fusion protein to the patient in an amount that is reduced by at least half of the amount of the dose of (i);
wherein the ActRIIA fusion protein comprises
(a) an ActRIIA polypeptide comprising an amino acid sequence that is at least 90% identical to an amino acid sequence corresponding to residues 30-110 of SEQ ID NO: 1;
(b) an Fc domain of an immunoglobulin;
(c) a linker domain positioned between the ActRIIA polypeptide domain and the Fc domain of the immunoglobulin.

66. A method of reducing a risk of telangiectasia when treating a patient with an ActRIIA fusion protein, the method comprising administering the ActRIIA fusion protein on a dosing regimen comprising:
(i) administering one or more doses of the ActRIIA fusion protein to a patient in an amount of 0.3 mg/kg once every 3 weeks for 24 weeks;
(ii) if the patient shows one or more symptoms or risk factors for developing telangiectasia, skipping a dose, wherein the patient is not treated for 6 weeks after step (i); and
(iii) resuming administering one or more doses of the ActRIIA fusion protein to the patient in an amount of 0.3 mg/kg once every 3 weeks for at least 24 weeks.

67. A method of decreasing a risk of telangiectasia when treating a patient with an ActRIIA fusion protein, the method comprising administering the ActRIIA fusion protein on a dosing regimen comprising:
(i) administering one or more doses of the ActRIIA fusion protein to a patient in an amount of 0.3 mg/kg once every 3 weeks for a period of 24 weeks or less;
(ii) after step (i), administering one or more doses of the ActRIIA fusion protein to the patient in an amount of 0.7 mg/kg once every 3 weeks for as long as the patient needs treatment; and
(iii) if the patient shows one or more symptoms or risk factors for developing telangiectasia, while receiving the dose of step (ii), administering one or more doses of the ActRIIA fusion protein, wherein the dose is administered in an amount of 0.3 mg/kg once every 3 weeks until symptoms improve;
wherein the ActRIIA fusion protein comprises an amino acid sequence that is at least 90% identical to an amino acid sequence corresponding to residues 30-110 of SEQ ID NO: 1, thereby decreasing the risk of telangiectasia.

68. The method of claim 67, wherein step (iii) comprises administering one or more doses of the ActRIIA fusion protein to the patient in an amount of 0.3 mg/kg once every 3 weeks for at least 24 weeks.

69. The method of claim 67, wherein the patient is not treated with the ActRIIA fusion protein for a period of at least 2-6 weeks between step (ii) and step (iii).

70. The method of claim 67, wherein the patient is not treated with the ActRIIA fusion protein for a period of at least 3 weeks between step (ii) and step (iii).

71. The method of claim 67, wherein, step (i) comprises administering the dose to the patient for at least 3 weeks.

72. The method of claim 67, wherein step (ii) comprises administering the dose to the patient for at least 21 weeks.

73. The method of claim 67, wherein step (ii) comprises administering the dose to the patient for at least 45 weeks.

74. The method of claim 65, wherein the one or more symptoms of telangiectasia are selected from the group consisting of pain, itching, threadlike red marks on the skin, mucocutaneous telangiectases, gastrointestinal bleeding, lesions on the skin, epistaxis, gingival bleeding, arteriovenous malformations, internal telangiectases, and red spots on the skin.

75. The method of claim 65, wherein the risk factors for developing telangiectasia are selected from the group consisting of:
i. low BMP9 levels;
ii. low BMP10 levels;
iii. low VEGF levels;
iv. hereditary hemorrhagic telangiectasia (HHT); and
v. connective tissue disease (CTD).

76. The method of claim 65, wherein the method alleviates or improves the severity of telangiectasia in the patient.

77. The method of claim 65, wherein the method prevents the progression of telangiectasia in the patient.

78. The method of claim 65, wherein the patient is receiving the ActRIIA fusion protein for the treatment of pulmonary hypertension or pulmonary arterial hypertension.

79. The method of claim 65, wherein the ActRIIA fusion protein comprises an amino acid sequence that is at least 95% identical to an amino acid sequence corresponding to residues 30-110 of SEQ ID NO: 1.

80. The method of claim 65, wherein the ActRIIA fusion protein comprises an amino acid sequence that is at least 95% identical to an amino acid sequence corresponding to residues 21-135 of SEQ ID NO: 1.

81. The method of claim 65, wherein the ActRIIA fusion protein comprises an amino acid sequence that is at least 98% identical to an amino acid sequence corresponding to residues 21-135 of SEQ ID NO: 1.

82. The method of claim 65, wherein the ActRIIA fusion protein comprises an amino acid sequence that is identical to the amino acid sequence of SEQ ID NO: 2.

83. The method of claim 65, wherein the Fc domain of the immunoglobulin is an Fc domain of an IgG1 immunoglobulin.

84. The method of claim 65, wherein the linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21).

85. The method of claim 65, wherein the ActRIIA fusion protein comprises an amino acid sequence that is at least 95% identical to the amino acid sequence of SEQ ID NO: 23.

86. The method of claim 65, wherein the ActRIIA fusion protein comprises an amino acid sequence that is at least 98% identical to the amino acid sequence of SEQ ID NO: 23.

87. The method of claim 65, wherein the ActRIIA fusion protein comprises an amino acid sequence that is identical to the amino acid sequence of SEQ ID NO: 23.

88. The method of claim 65, wherein the ActRII fusion protein comprises an amino acid sequence that is identical to the amino acid sequence of SEQ ID NO: 40.


SEQUENCE LISTING (40 sequences)

SEQ ID NO: 1 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 2 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 3 - protein, 100 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 4 - DNA, 1539 bp, Homo sapiens
  source 1..1539: mol_type = genomic DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 5 - DNA, 345 bp, Homo sapiens
  source 1..345: mol_type = genomic DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 6 - protein, 115 aa, Homo sapiens
  VARIANT 45: note = Any amino acid
ILGRSETQECLFFNANWEKDRTNQTQVEPCYGDKDKRRHCFATWXNISGSIEIVKQGCWLDDINCYDRTDCVEKKDSPEVYFCCCEGNMCNEKFSTFPEMEVTQPTSNPVTPKPP

SEQ ID NO: 7 - protein, 115 aa, Ovis aries
  VARIANT 45: note = Any amino acid
  VARIANT 114: note = Any amino acid
ILGRSETQECLFYNANWERDRTNRTGVESCYGDKDKRRHCFATWXNISGSIDIVKQGCWLDDINCYDRTDCIEKKDSPEVYFCCCEGNMCNERFSTFPEMEVTQPTSNPVTPKXP

SEQ ID NO: 8 - protein, 115 aa, unidentified
  VARIANT 45: note = Any amino acid
  VARIANT 113: note = Any amino acid
ILGRSETQECLFFNANWERDRTNQTGVEPCYGDKDKHRHCFATWXNISGSIEIVKQGCWLDDINCYDRTDCIEKKDSPEVYFCCCEGNMCNEKFSTFPEMEVTQPTSNPVTPXAP

SEQ ID NO: 9 - protein, 115 aa, Mus sp.
  VARIANT 45: note = Any amino acid
  VARIANT 68: note = Any amino acid
ILGRSETQECLFFNANWERDRTNQTGVEPCYGDKDKRRHCFATWXNISGSIEIVKQGCWLDDINCYDXTDCIEKKDSPEVYFCCCEGNMCNEKFSTFPEMEVTQPTSNPVTPKPP

SEQ ID NO: 10 - protein, 115 aa, Gallus gallus
  VARIANT 45: note = Any amino acid
  VARIANT 113: note = Any amino acid
ILGRSETQECLYYNANWEKDKTNRSGIEPCYGDKDKRRHCFATWXNISGSIEIVKQGCWLDDINCYDRNDCIEKKDSPEVFFCCCEGNMCNERFFTFPEMEVTQPTSNPVTPXPP

SEQ ID NO: 11 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - skipped

SEQ ID NO: 17 - protein, 4 aa, synthetic construct
GGGG

SEQ ID NO: 18 - protein, 5 aa, synthetic construct
TGGGG

SEQ ID NO: 19 - protein, 5 aa, synthetic construct
SGGGG

SEQ ID NO: 20 - protein, 4 aa, synthetic construct
TGGG

SEQ ID NO: 21 - protein, 4 aa, synthetic construct
SGGG

SEQ ID NO: 22 - protein, 5 aa, synthetic construct
GGGGS

SEQ ID NO: 23 - protein, 344 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 21 aa, Apis cerana
MKFLVNVALVFMVVYISYIYA

SEQ ID NO: 25 - protein, 22 aa, unidentified
MDAMKRGLCCVLLLCGAVFVSP

SEQ ID NO: 26 - protein, 20 aa, unidentified
MGAAAKLAFAVFLISCSSGA

SEQ ID NO: 27 - protein, 369 aa, synthetic construct
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1114 bp, synthetic construct
  source 1..1114: mol_type = other DNA
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 29 - protein, 9 aa, synthetic construct
ILGRSETQE

SEQ ID NO: 30 - protein, 329 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 32 - protein, 225 aa, Homo sapiens; identical to SEQ ID NO: 11

SEQ ID NO: 33 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVLHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPGK

SEQ ID NO: 35 - protein, 229 aa, Homo sapiens; identical to SEQ ID NO: 15

SEQ ID NO: 36 - protein, 115 aa, Bos taurus
  VARIANT 45: note = Any amino acid
  VARIANT 113: note = Any amino acid
ILGRSETQECLFYNANWERDRTNRTGVESCYGDKDKRRHCFATWXNISGSIEIVKQGCWLDDINCYDRTDCIEKKDSPEVYFCCCEGNMCNERFSTFPEMEVTQPTSNPVTPXPP

SEQ ID NO: 37 - protein, 115 aa, Tyto alba; identical to SEQ ID NO: 10
  VARIANT 45: note = Any amino acid
  VARIANT 113: note = Any amino acid

SEQ ID NO: 38 - protein, 115 aa, Myotis davidii
  VARIANT 45: note = Any amino acid
ILGRSETQECLFYNANWERDKTNRTGVELCYGDKDKHRHCFATWXNISGSIEIVKQGCWLDDINCYDKTDCIEKKDSPEVYFCCCEGNMCNERFSTFPEMEVTQPTSNPVTPKPP

SEQ ID NO: 39 - protein, 6 aa, synthetic construct
HHHHHH

SEQ ID NO: 40 - protein, 343 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG